(19) United States
(12) Patent Application Publication
(10) Pub. No.: US 2026/0240858 A1
(43) Pub. Date: Aug. 20, 2026

(54) ORAL FORMULATIONS, METHODS OF MANUFACTURE AND USES THEREOF

(71) Applicant: Agency For Science, Technology And Research, Connexis North Tower (SG)

(72) Inventors: Kanthraj ETHIRAJULU, Chromos (SG); Vimala Ranjani NELLORE, Chromos (SG)

(73) Assignee: Agency For Science, Technology And Research, Connexis North Tower (SG)

(21) Appl. No.: 17/612,894

(22) PCT Filed: May 18, 2020
(86) PCT No.: PCT/SG2020/050289

§ 371 Date: Nov. 19, 2021

(30) Foreign Application Priority Data
May 22, 2019 (SG) 10201904586U

(51) Int. Cl. A61K 31/522; A61K 9/48

(52) U.S. Cl. CPC A61K 31/522; A61K 9/4858

(57) ABSTRACT

The invention relates generally to oral formulations and methods of manufacture thereof. In particular, the invention relates capsule formulations of a specific compound for treating diseases and/or disorders related to the Wnt pathway. The capsule formulation comprises the specific compound or a pharmaceutically acceptable salt or solvate thereof, at least one excipient and sodium lauryl sulphate.

### FIELD OF INVENTION

The invention relates generally to oral formulations and methods of manufacture thereof. In particular, the invention relates capsule formulations of a specific compound for treating diseases and/or disorders related to the Wnt pathway.

### BACKGROUND

The process of formulating a pharmaceutical product is a process in which different chemical substances, including the active drug, are combined to produce a final medicinal product. As is known in the art, formulation studies are not straightforward and involve much understanding and research to develop a preparation of the drug which is both stable, bioavailable and effective, physiologically compatible with the patient. It is commonly known that small changes in a formulation can lead to unpredictable results and that the most significant challenges and considerations when developing a drug formulation are safety, appropriate therapeutic and delivery profiles, bioavailability, stability and solubility.

For example, orally administered drugs need to be formulated such that the drug itself does not adversely react with the excipients and that the drug is compatible with these other substances in a way that does not cause harm, whether direct or indirect.

A drug's physical, chemical, and mechanical properties have to also be considered in order to choose what other ingredients (excipients) should be used in the preparation. Further, the solution behaviour of a drug under a variety of stress conditions such as freeze/thaw, temperature, pH, shear stress among others needs to be studied to identify mechanisms of degradation.

Factors such as particle size, polymorphism, pH, and solubility can influence bioavailability and hence the activity of a drug. The drug must be combined with inactive ingredients by a method which ensures that the quantity of drug present is consistent in each dosage unit. For customer appeal and good manufacturing practices, the final product must have a uniform appearance, an acceptable taste, tablet hardness, and/or capsule disintegration.

The selection of the physical medium that an oral formulation is to be delivered is also of consideration. For example, the mechanism of action of an active ingredient and how the active ingredient is intended to be delivered need to be considered as it is believed that active ingredients from capsules are absorbed differently than from tablets. Each type of oral formulation has its own advantages and disadvantages, and the formulation scientist must balance these factors to get to an acceptable delivery form. In this regard, while capsules can be good oxygen barriers, provide protection for sensitive ingredients and reduced gastrointestinal irritation, the ingredients can interact with capsule shell, is limited to certain fill weight based on capsule volumes and the pH range of the formulation must be strictly controlled. On the other hand, tablets typically have lower cost, is the preferred delivery for products with large amounts per serving due to compressibility, can be consumed by multiple demographics and provide better dissolution control for quick, delayed, or extended release. However, it has potentially poor disintegration in the GI tract, is susceptible to heat and moisture and is potentially sensitive to the coatings.

The formulation scientist also needs to take into account the release profile of the drug from the formulation, which can depend on the excipients used and how the formulation is delivered. Through the control of the release profile, the functionality of the drug can be improved, the side effects can be at least reduced and better patient compliance can be achieved.

Even if a formulation scientist has overcome all the above mentioned problems, the formulation needs to pass the hurdle of clinical trials. In this regard, consideration is given to drug loading, which is the ratio of the active drug to the total contents of the dose. A low drug load may cause homogeneity problems while a high drug load may pose flow problems or require large capsules if the compound has a low bulk density.

The stability of the formulation and the drug also needs to be investigated and conditions must be developed to ensure that the drug is stable in the preparation and has an acceptable shelf-life. If the drug is unstable in the formulation, it will invalidate the results from clinical trials since it would be impossible to know what the administered dose actually was.

Accordingly, it is generally desirable to produce a formulation which overcomes or ameliorate one or more of the above mentioned difficulties and is effective.

### SUMMARY

The present invention provides an oral formulation, comprising:
a) a compound of Formula (I) having the following structure:

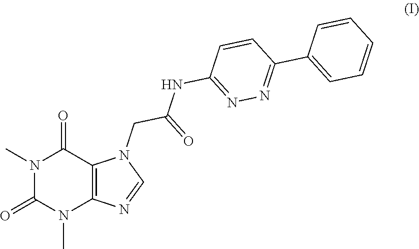 [SMILES: Cn1c(=O)c2c(ncn2CC(=O)Nc2ccc(-c3ccccc3)nn2)n(C)c1=O]

or a pharmaceutically acceptable salt or solvate thereof;
b) at least one excipient selected from the group consisting of starch, mannitol, dicalcium phosphate and microcrystalline cellulose; and
c) sodium lauryl sulphate;
   wherein the oral formulation is provided as a capsule.

In some embodiments, the at least one excipient is from about 70% w/w to about 99% w/w.

In some embodiments, the at least one excipient is mannitol or starch.

In some embodiments, the compound of Formula (I) is from about 0.5% w/w to about 27% w/w.

In some embodiments, sodium lauryl sulphate is from about 0.5% w/w to about 3% w/w.

In some embodiments, the oral formulation further comprises magnesium stearate.

In some embodiments, the magnesium stearate is from about 0.5% w/w to about 2% w/w.

The present invention also provides an oral formulation, comprising:
a) a compound of Formula (I) having the structure:

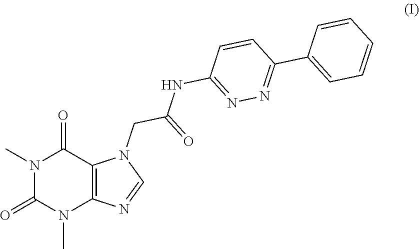 [SMILES: Cn1c(=O)c2c(ncn2CC(=O)Nc2ccc(-c3ccccc3)nn2)n(C)c1=O]

or a pharmaceutically acceptable salt or solvate thereof from about 0.5% w/w to about 27% w/w;
b) mannitol from about 69% w/w to about 98.5% w/w;
c) sodium lauryl sulphate from about 0.5% w/w to about 2% w/w; and
d) magnesium stearate from about 0.5% w/w to about 2% w/w;
   wherein the oral formulation is provided as a capsule.

The present invention also provides an oral formulation, comprising:
a) a compound of Formula (I) having the structure:

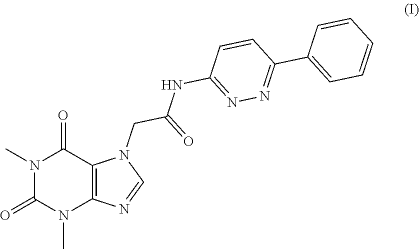 [SMILES: Cn1c(=O)c2c(ncn2CC(=O)Nc2ccc(-c3ccccc3)nn2)n(C)c1=O]

or a pharmaceutically acceptable salt or solvate thereof from about 0.5% w/w to about 27% w/w;
b) starch from about 69% w/w to about 98.5% w/w;
c) sodium lauryl sulphate from about 0.5% w/w to about 2% w/w; and
d) magnesium stearate from about 0.5% w/w to about 2% w/w;
   wherein the oral formulation is provided as a capsule.

The present invention also provides a process for manufacturing an oral formulation as disclosed herein, including the steps of:
i) mixing compound of Formula (I), the at least one excipient and sodium lauryl sulphate to form a mixture; and
ii) encapsulating the mixture in a capsule to form the oral formulation.

In some embodiments, the mixing step is selected from direct blending or trituration.

In some embodiments, the process further includes a step (ia) after step (i) of mixing magnesium stearate into the mixture.

In some embodiments, the encapsulating step includes encapsulating the mixture into gelatin capsules.

The present invention also provides a method of treating a proliferative disease or disorder associated with Wnt pathway in a subject in need thereof, comprising administering an oral formulation as disclosed herein to the subject.

The present invention also provides an oral formulation for use in the treatment of a proliferative disease or disorder associated with Wnt pathway in a subject in need thereof, comprising administering an oral formulation as disclosed herein to the subject.

The present invention also provides a use of an oral formulation in the manufacture of a medicament for the treatment of a proliferative disease or disorder associated with Wnt pathway, comprising administering an oral formulation as disclosed herein.

In an embodiment, the proliferative disease or condition is selected from the group consisting of cancer, fibrosis, stem cell and diabetic retinopathy, rheumatoid arthritis, psoriasis and myocardial infarction.

In an embodiment, the proliferative disease or condition is selected from the group consisting of cervical cancer, colon cancer, breast cancer, bladder cancer, head and neck cancer, gastric cancer, lung cancer, ovarian cancer, prostate cancer, thyroid cancer, non-small-cell lung cancer, chronic lymphocytic leukemia, mesothelioma, melanoma, pancreatic adenocarcinoma, basal cell carcinoma, osteosarcoma, hepatocellular carcinoma, Wilm's tumour, medulloblastoma, pulmonary fibrosis, liver fibrosis, skin fibrosis, renal fibrosis, stem cell and diabetic retinopathy, rheumatoid arthritis, psoriasis and myocardial infarction.

### BRIEF DESCRIPTION OF DRAWINGS

Embodiments of the present invention are hereafter described, by way of non-limiting example only, with reference to the accompanying drawings in which:

FIG. 1 illustrates an example of the brim filling and the finished capsules

FIG. 2 plots the dissolution of 20 mg API (free base) capsules over time in 1% SLS in 0.1M HCl at 50 RPM

FIG. 3 plots the dissolution of 20 mg API (free base) capsules over time in 2% SLS in 0.1M HCl at 50 and 75 RPM

FIG. 4 plots the dissolution of 1 mg API (free base) capsules over time in 1% SLS in 0.1M HCl at 75 RPM

FIG. 5 plots the dissolution of 1 mg API (free base) capsules over time in 2% SLS in 0.1M HCl at 75 RPM

FIG. 6 illustrates the process for preparing an oral formulation using trituration

FIG. 7 illustrates the process for preparing an oral formulation using blending

FIG. 8 plots the dissolution of 1 mg API (with starch) capsules over time at an initial time point (immediately after formulation)

FIG. 9 plots the dissolution of 1 mg API (with starch) capsules over time after storage for 1 month at 25° C. and 60% relative humidity

FIG. 10 plots the dissolution of 1 mg API (with starch) capsules over time after storage for 1 month at 40° C. and 75% relative humidity

FIG. 11 compares the dissolution profile of 1 mg API (with starch) at three different conditions

FIG. 12 plots the dissolution of 1 mg API (with starch) capsules over time after storage for 3 month at 25° C. and 60% relative humidity

FIG. 13 plots the dissolution of 1 mg API (with starch) capsules over time after storage for 3 month at 40° C. and 75% relative humidity

FIG. 14 plots the dissolution of 1 mg API (with starch) capsules over time after storage for 6 month at 25° C. and 60% relative humidity

FIG. 15 plots the dissolution of 1 mg API (with starch) capsules over time after storage for 6 month at 40° C. and 75% relative humidity

FIG. 16 compares the dissolution profile of 1 mg API (with starch) at three different conditions and at three different time points

FIG. 17 plots the dissolution of 1 mg API (with mannitol) capsules over time at an initial time point (immediately after formulation)

FIG. 18 plots the dissolution of 1 mg API (with mannitol) capsules over time after storage for 1 month at 25° C. and 60% relative humidity

FIG. 19 plots the dissolution of 1 mg API (with mannitol) capsules over time after storage for 1 month at 40° C. and 75% relative humidity

FIG. 20 compares the dissolution profile of 1 mg API (with mannitol) at three different conditions

FIG. 21 plots the dissolution of 20 mg API (with starch) capsules over time at an initial time point (immediately after formulation)

FIG. 22 plots the dissolution of 20 mg API (with starch) capsules over time after storage for 1 month at 25° C. and 60% relative humidity

FIG. 23 plots the dissolution of 20 mg API (with starch) capsules over time after storage for 1 month at 40° C. and 75% relative humidity

FIG. 24 compares the dissolution profile of 20 mg API (with starch) at three different conditions

FIG. 25 plots the dissolution of 20 mg API (with starch) capsules over time after storage for 3 month at 25° C. and 60% relative humidity

FIG. 26 plots the dissolution of 20 mg API (with starch) capsules over time after storage for 3 month at 40° C. and 75% relative humidity

FIG. 27 plots the dissolution of 20 mg API (with starch) capsules over time after storage for 6 month at 25° C. and 60% relative humidity

FIG. 28 plots the dissolution of 20 mg API (with starch) capsules over time after storage for 6 month at 40° C. and 75% relative humidity

FIG. 29 compares the dissolution profile of 20 mg API (with starch) at three different conditions and at three different time points

FIG. 30 plots the dissolution of 20 mg API (with mannitol) capsules over time at an initial time point (immediately after formulation)

FIG. 31 plots the dissolution of 20 mg API (with mannitol) capsules over time after storage for 1 month at 25° C. and 60% relative humidity

FIG. 32 plots the dissolution of 20 mg API (with mannitol) capsules over time after storage for 1 month at 40° C. and 75% relative humidity

FIG. 33 compares the dissolution profile of 20 mg API (with mannitol) at three different conditions

### DETAILED DESCRIPTION

The present invention is predicated on the discovery that certain capsule based oral formulations comprising a compound of Formula (I) are advantageous. This is based on the understanding that the compound of Formula (I) has certain physical and chemical properties (as well as pharmacokinetic properties) that has made the formulation process challenging. For example, the compound of Formula (I) was found to have low aqueous solubility, permeability and poor flow properties. While a tablet is usually easier to compound, the inventors have found that by compounding compound of Formula (I) with certain excipients to form an oral capsule formulation, the flow properties can be improved. Further, the oral capsule formulations have good wettability and dissolution profile, allowing for a good bioavailability.

In particular, the compound of Formula (I) does not appear to be stable and is also sensitive to degradation. Without wanting to be bound by theory, it is believed that this is due to solid state hydrolysis. Hydrolysis can occur at the amide position of compound of Formula (I) with water acting as a base. Due to the greater electronegativity of oxygen atom, the carbonyl group is polarized such that the oxygen atom carries a partial negative charge while the carbon atom carries a partial positive charge. As a result, the carbonyl group can be attacked nucleophilically. Based on this understanding, it is believed that it will be very difficult to reliably formulate the pharmaceutically active substance into a tablet as the amount of substance to be added to provide the same dosage will vary greatly depending upon the degree of hydrolysis. Furthermore variations in hydration or solid form (“polymorphism”) can lead to changes in physico-chemical properties, such as solubility or dissolution rate, which might in turn lead to inconsistent oral absorption in a patient. Additionally, the compound of Formula (I) has a low solubility, at less than 6 μg/mL in water. It is also believed that the act of compacting the compound of Formula (I) as a tablet will compound this issue. Accordingly, a capsule form can be particularly advantageous over that of a tablet form.

The oral formulation comprises a compound of Formula (I) having the following structure:

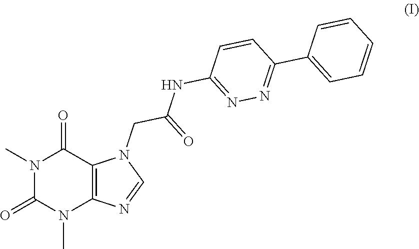 [SMILES: Cn1c(=O)c2c(ncn2CC(=O)Nc2ccc(-c3ccccc3)nn2)n(C)c1=O]

or a pharmaceutically acceptable salt or solvate thereof.

In some embodiments, compound of Formula (I) is from about 0.5% w/w to about 27% w/w. In other embodiments, compound of Formula (I) is from about 1% w/w to about 25% w/w, or from about 1% w/w to about 20% w/w.

As compound of Formula (I) has a low solubility in water, the inventors have found that it is further advantageous to have the compound at a certain particle size, which is helpful in obtaining a desirable dissolution profile of the formulation. In some embodiments, compound of Formula (I) has a mean particle size of less than about 1000 μm, less than about 900 μm, less than about 800 μm, less than about 700 μm, less than about 600 μm, less than about 500 μm, less than about 400 μm, less than about 350 μm, less than about 300 μm, less than about 250 μm, less than about 200 μm, less than about 150 μm or less than about 100 μm. In other embodiments, compound of Formula (I) has a mean particle size of less than 100 μm, 90 μm, 80 μm, 70 μm, 60 μm, 50 μm, 40 μm, 30 μm, or 20 μm. In other embodiments, compound of Formula (I) has a mean particle size of about 100 μm, 90 μm, 80 μm, 70 μm, 60 μm, 50 μm, 40 μm, 30 μm, or 20 μm.

An excipient is a substance that is added to the active ingredient of a formulation. Excipients can be added for the purpose of long-term stabilization, bulking up solid formulations that contain potent active ingredients in small amounts, or to confer a therapeutic enhancement on the active ingredient in the final dosage form, such as facilitating drug absorption, reducing viscosity, or enhancing solubility. For example, excipients can be added to a formulation to significantly affect the chemical and physical properties of the formulation and thus affecting the biopharmaceutical profile. Excipients can also be useful in the manufacturing process, to aid in the handling of the active substance concerned such as by facilitating powder flowability or non-stick properties, in addition to aiding in vitro stability such as prevention of denaturation or aggregation over the expected shelf life. The selection of appropriate excipients also depends upon the route of administration and the dosage form, as well as the active ingredient and other factors. The inventors have found that not all excipients can be used with compound of Formula (I). In particular, the inventors have found that excipients with the ability to form hydrogen bonds are advantageous for use with compound of Formula (I). It is believed that the OH groups and O⁻ groups can form favourable electrostatic interactions with the HN, N and O in compound of Formula (I), thereby allowing for the excipient to be closely associated with compound of Formula (I) for improved chemical and physical properties of the formulation. In this regard, when formulated with such excipients, it is shown that the formulations have good flow properties and good stability over time at high temperature and high relative humidity. The excipients also allows for good manufacturability of the oral formulation. In this regard, when formed as capsules, the capsules are easy to fill to the brim by tamping. There is an acceptable rejection rate of formulated capsules. Further, the use of these excipients in an oral formulation allows for the absorption of water, forming a viscous liquid or paste in the gastrointestinal tract. This helps in improving the wettability and dissolution of compound of Formula (I), which is poorly wettable, for the compound to be more easily absorbed. For capsules containing 1 mg or 20 mg of compound of Formula (I), dissolution is shown to be at least 85% after 15 minutes. A further advantage of these excipients is that they attract water molecules away from the active ingredient, and hence can improve the shelf-life of the formulation as the active ingredient is less prone to degradation.

In some embodiments, the at least one excipient is selected from the group consisting of starch, mannitol, dicalcium phosphate and microcrystalline cellulose. In some embodiments, the at least one excipient is selected from the group consisting of pregelatinised starch, partially pregelatinised starch mannitol, dicalcium phosphate anhydrous and microcrystalline cellulose. In other embodiments, the at least one excipient is selected from starch and mannitol. In other embodiments, the at least one excipient is pregelatinised starch. In other embodiments, the at least one excipient is starch 1500. In other embodiments, the at least one excipient is mannitol.

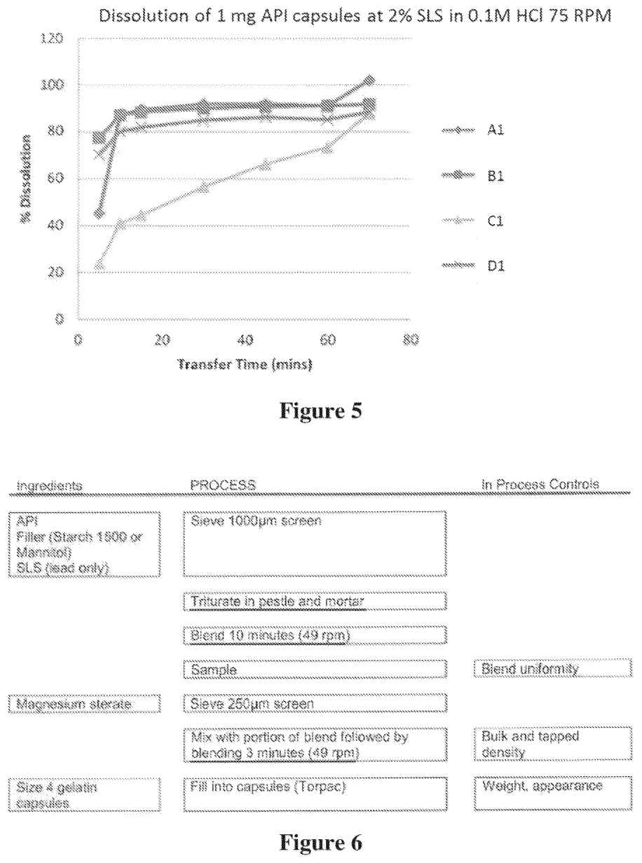

Out of the 4 excipients, 2 of the excipients (starch and mannitol) were found to provide a particular advantage (over that of dicalcium phosphate and microcrystalline cellulose). For example, as shown in FIGS. 4 and 5, the dissolution profile of compound of Formula (I) with starch or mannitol was at least about 80% at 20 min while microcrystalline cellulose (Avicel PH 102) was only about 40% at 20 min.

Starch is a polysaccharide comprising glucose monomers joined in α 1,4 linkages. The simplest form of starch is the linear polymer amylose; amylopectin is the branched form. When a starch is pre-cooked, it can then be used to thicken instantly in cold water. This is referred to as a pregelatinised starch. This process breaks down the intermolecular bonds of starch molecules in the presence of water and heat, allowing the hydrogen bonding sites (the hydroxyl hydrogen and oxygen) to engage more water. This irreversibly dissolves the starch granule in water. The cooked starch is then dried to obtain pregelatinised starch. Accordingly, partially pregelatinised starch is starch that has undergone only partial cooking.

Advantageously, the formulation with starch was found to be stable for at least 3 years.

Mannitol is a polyol (sugar alcohol) (C₆H₈(OH)₆) and an isomer of sorbitol. Mannitol is industrially derived from the sugar fructose, and is roughly half as sweet as sucrose. Mannitol has a cooling effect often used to mask bitter tastes, and may be used in gums and candies. Mannitol is also found naturally in many species, including plants, bacteria, and fungi. Pearlitol 200SD is an example of mannitol that can be purchased.

Dicalcium phosphate is the calcium phosphate with the formula CaHPO₄ and its dihydrate. Dicalcium phosphate anhydrous refers to the anhydrous form of dicalcium phosphate; i.e. contains no water.

Microcrystalline cellulose (MCC) is a term for refined wood pulp. It is a naturally occurring polymer and is composed of glucose units connected by a 1-4 beta glycosidic bond. MCC is prepared by acid hydrolysis of cellulose using 2 M hydrochloric acid at 105° C. for 15 min. The highly reactive amorphous regions selectively hydrolyze, releasing the crystallites, which are subsequently mechanically dispersed.

In some embodiments, the at least one excipient is from about 70% w/w to about 99% w/w. In other embodiments, the at least one excipient is from about 71% w/w to about 99% w/w, or from about 72% w/w to about 99% w/w, or from about 73% w/w to about 99% w/w, or from about 75% w/w to about 99% w/w, or from about 76% w/w to about 99% w/w, or from about 77% w/w to about 99% w/w, or from about 78% w/w to about 99% w/w, or from about 79% w/w to about 99% w/w, or from about 80% w/w to about 99% w/w.

Accordingly, in an embodiment, the oral formulation comprises:
a) a compound of Formula (I) having the following structure:

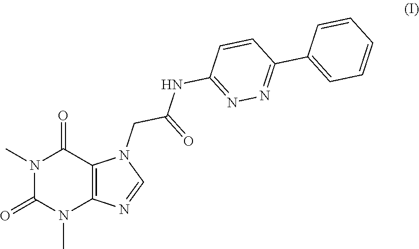 [SMILES: Cn1c(=O)c2c(ncn2CC(=O)Nc2ccc(-c3ccccc3)nn2)n(C)c1=O]

or a pharmaceutically acceptable salt or solvate thereof;
b) at least one excipient selected from the group consisting of starch and mannitol; and
c) sodium lauryl sulphate (SLS);
   wherein the oral formulation is provided as a capsule.

A further advantage of the capsule form over the tablet form is the lack of compacting the formulation. It was found that by maintaining the formulation in a loose form and protected within a capsule, the stability of the compound of Formula (I) can be maintained for a longer period of time.

Sodium lauryl sulphate (SLS) is an anionic surfactant commonly employed in a wide range of non-parenteral pharmaceutical formulations. It is a detergent and wetting agent effective in both alkaline and acidic conditions. The inventors have found that adding SLS to the formulation can further improve the wettability of compound of Formula (I), and thereby further improve its dissolution.

Advantageously, the inventors have found that SLS can increase the solubility of the compound of Formula (I) by at least 50 times. In comparison, tests with other surfactants such as Polysorbate 80 (Tween 80), Polyvinylpyrrolidone (PVP) or Poloxamer did not provide such an advantage.

In some embodiments, SLS increases the solubility of compound of Formula (I) by at least 55 time, 60 times, 65 times, 70 times, 75 times, 80 times, 85 times, 90 times, 95 times or 100 times.

In some embodiments, sodium lauryl sulphate is from about 0.5% w/w to about 3% w/w. In other embodiments, SLS is from about 1% w/w to about 3% w/w, or about 1% w/w to about 2% w/w.

In some embodiments, the oral formulation comprises:
a) a compound of Formula (I) or a pharmaceutically acceptable salt or solvate thereof from about 0.5% w/w to about 27% w/w;
b) at least one excipient selected from the group consisting of starch, mannitol, dicalcium phosphate and microcrystalline cellulose from about 70% w/w to about 99% w/w; and
c) sodium lauryl sulphate from about 0.5% w/w to about 3% w/w;
wherein the oral formulation is provided as a capsule.

In some embodiments, the oral formulation comprises:
a) a compound of Formula (I) or a pharmaceutically acceptable salt or solvate thereof from about 0.5% w/w to about 27% w/w;
b) at least one excipient selected from the group consisting of starch and mannitol from about 70% w/w to about 99% w/w; and
c) sodium lauryl sulphate from about 0.5% w/w to about 3% w/w;
wherein the oral formulation is provided as a capsule.

In some embodiments, the oral formulation comprises:
a) a compound of Formula (I) or a pharmaceutically acceptable salt or solvate thereof from about 0.5% w/w to about 20% w/w;
b) at least one excipient selected from the group consisting of starch, mannitol, dicalcium phosphate and microcrystalline cellulose from about 78% w/w to about 99% w/w; and
c) sodium lauryl sulphate from about 0.5% w/w to about 2% w/w;
wherein the oral formulation is provided as a capsule.

In some embodiments, the oral formulation comprises:
a) a compound of Formula (I) or a pharmaceutically acceptable salt or solvate thereof from about 0.5% w/w to about 20% w/w;
b) at least one excipient selected from the group consisting of starch and mannitol from about 78% w/w to about 99% w/w; and
c) sodium lauryl sulphate from about 0.5% w/w to about 2% w/w;
wherein the oral formulation is provided as a capsule.

In some embodiments, the oral formulation further comprises magnesium stearate. Magnesium stearate prevents sticking of the formulation to the instrumentations.

In some embodiments, the magnesium stearate is from about 0.5% w/w to about 2% w/w. In other embodiments, magnesium stearate is from about 1% w/w to about 2% w/w.

Accordingly, the present invention provides an oral formulation, comprising:
a) a compound of Formula (I) having the structure:

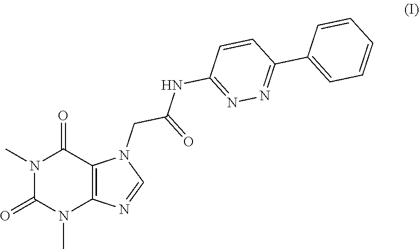 [SMILES: Cn1c(=O)c2c(ncn2CC(=O)Nc2ccc(-c3ccccc3)nn2)n(C)c1=O]

or a pharmaceutically acceptable salt or solvate thereof from about 0.5% w/w to about 27% w/w;
b) mannitol from about 69% w/w to about 98.5% w/w;
c) sodium lauryl sulphate from about 0.5% w/w to about 2% w/w; and
d) magnesium stearate from about 0.5% w/w to about 2% w/w;
   wherein the oral formulation is provided as a capsule.

In some embodiments, the oral formulation comprises:
a) a compound of Formula (I) having the structure:

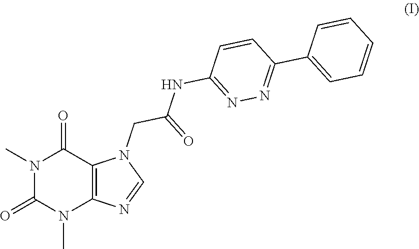 [SMILES: Cn1c(=O)c2c(ncn2CC(=O)Nc2ccc(-c3ccccc3)nn2)n(C)c1=O]

or a pharmaceutically acceptable salt or solvate thereof from about 0.5% w/w to about 20% w/w;
b) mannitol from about 78% w/w to about 97.5% w/w;
c) sodium lauryl sulphate at about 1% w/w; and
d) magnesium stearate at about 1% w/w;
   wherein the oral formulation is provided as a capsule.

The present invention also provides an oral formulation, comprising:
a) a compound of Formula (I) having the structure:

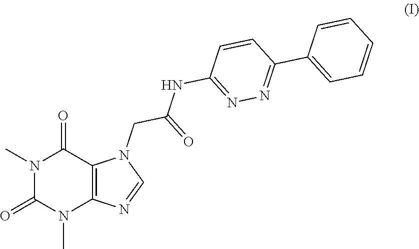 [SMILES: Cn1c(=O)c2c(ncn2CC(=O)Nc2ccc(-c3ccccc3)nn2)n(C)c1=O]

or a pharmaceutically acceptable salt or solvate thereof from about 0.5% w/w to about 27% w/w;
b) starch from about 69% w/w to about 98.5% w/w;
c) sodium lauryl sulphate from about 0.5% w/w to about 2% w/w; and
d) magnesium stearate from about 0.5% w/w to about 2% w/w;
   wherein the oral formulation is provided as a capsule.

In some embodiments, an oral formulation, comprising:
a) a compound of Formula (I) having the structure:

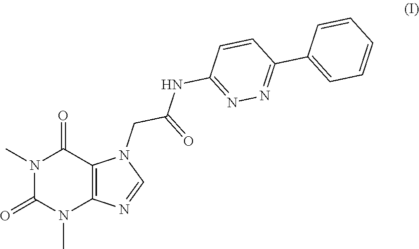 [SMILES: Cn1c(=O)c2c(ncn2CC(=O)Nc2ccc(-c3ccccc3)nn2)n(C)c1=O]

or a pharmaceutically acceptable salt or solvate thereof from about 0.5% w/w to about 20% w/w;
b) starch from about 78% w/w to about 97.5% w/w;
c) sodium lauryl sulphate at about 1% w/w; and
d) magnesium stearate at about 1% w/w;
   wherein the oral formulation is provided as a capsule.

In some embodiments, an oral formulation, comprising:
a) a compound of Formula (I) having the structure:

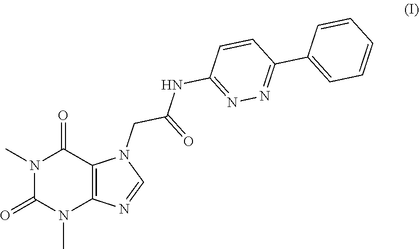 [SMILES: Cn1c(=O)c2c(ncn2CC(=O)Nc2ccc(-c3ccccc3)nn2)n(C)c1=O]

or a pharmaceutically acceptable salt or solvate thereof from about 0.5% w/w to about 20% w/w;
b) starch from about 78% w/w to about 97.5% w/w;
c) sodium lauryl sulphate at about 1% w/w; and
d) magnesium stearate at about 1% w/w;
   wherein the oral formulation is provided as a capsule; and
   wherein compound of Formula (I) has a mean particle size of less than about 1000 μm.

The present invention also provides a process for manufacturing an oral formulation as disclosed herein, including the steps of:
i) mixing compound of Formula (I), the at least one excipient and sodium lauryl sulphate to form a mixture; and
ii) encapsulating the mixture in a capsule to form the oral formulation.

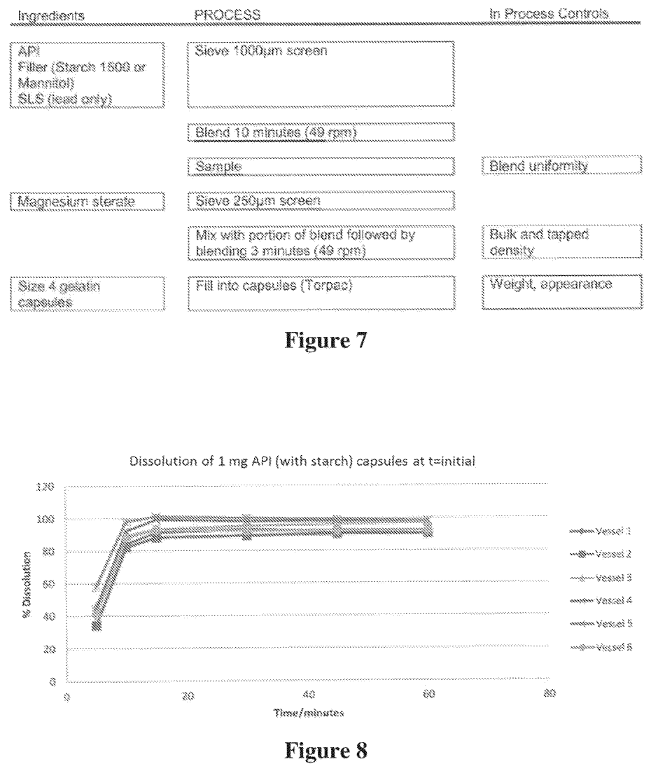

As shown in FIGS. 6 and 7, before the mixing step, compound of Formula (I) and the at least one excipient can be first sieved such that an appropriate particle sizing is obtained. The sieve can be a 1000 μm sieve. This allows for better flow property control and consistency between batches. It is further advantageous to have an appropriate particle size as it assist in the dissolution profile of the formulation. In other embodiments, the sieve is 900 μm, 800 μm, 700 μm, 600 μm, 500 μm, 400 μm, 350 μm, 300 μm, 250 μm, 200 μm, 150 μm or 100 μm.

In some embodiments, the mixing step is selected from direct blending or trituration.

In some embodiments, the process further includes a step (ia) after step (i) of mixing magnesium stearate into the mixture. Advantageously, magnesium stearate prevents or at least reduces the stickiness of the formulation to the equipment, and accordingly allows for good recovery of the formulation.

In some embodiments, the magnesium stearate is screened before being mixed with the mixture. In this regard, magnesium stearate may be sieved to obtain a suitable particle size. The screening is done to remove lumps/aggregates to ensure its free distribution during the subsequent blending step. It also ensures the consistency of the formulation and that the desired effect provided by the inclusion of the excipient is homogenously dispersed within the formulation. In some embodiments, magnesium stearate is screened by passing it through a 350 μm sieve, or a 300 μm sieve, or a 250 μm sieve, or a 200 μm sieve, or a 150 μm sieve, or a 100 μm sieve. In some embodiments, magnesium stearate has a mean particle size of about 100 μm, about 150 μm, about 200 μm, about 250 μm, about 300 μm, about 350 μm, or about 400 μm.

In some embodiments, the encapsulating step includes encapsulating the mixture into capsules. In some embodiments, the mixture is encapsulated into gelatin capsules, hydroxypropyl methylcellulose (HPMC) capsules, starch capsules, pullulan capsules or polyvinyl acetate (PVA) capsules. In some embodiments, the capsules are gelatin capsules. In some embodiments, size 4 gelatin capsules are used to encapsulate the mixture.

The present invention also provides a method of treating a proliferative disease or disorder associated with Wnt pathway, comprising administering an oral formulation as disclosed herein.

The present invention also provides an oral formulation for use in the treatment of a proliferative disease or disorder associated with Wnt pathway, comprising administering an oral formulation as disclosed herein.

The present invention also provides a use of an oral formulation in the manufacture of a medicament for the treatment of a proliferative disease or disorder associated with Wnt pathway, comprising administering an oral formulation as disclosed herein.

In an embodiment, the proliferative disease or disorder is associated with the up-regulation of the Wnt pathway.

In an embodiment, the proliferative disease or condition is selected from the group consisting of cancer, fibrosis, stem cell and diabetic retinopathy, rheumatoid arthritis, psoriasis and myocardial infarction.

In an embodiment, the proliferative disease or condition is selected from the group consisting of cervical cancer, colon cancer, breast cancer, bladder cancer, head and neck cancer, gastric cancer, lung cancer, ovarian cancer, prostate cancer, thyroid cancer, non-small-cell lung cancer, chronic lymphocytic leukemia, mesothelioma, melanoma, pancreatic adenocarcinoma, basal cell carcinoma, osteosarcoma, hepatocellular carcinoma, Wilm's tumour, medulloblastoma, pulmonary fibrosis, liver fibrosis, skin fibrosis, renal fibrosis, stem cell and diabetic retinopathy, rheumatoid arthritis, psoriasis and myocardial infarction.

The compound of Formula (I) can be formulated or administered to a subject as a pharmaceutically acceptable salt thereof. Suitable pharmaceutically acceptable salts include, but are not limited to salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benezenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium. In particular, the present invention includes within its scope cationic salts eg sodium or potassium salts, or alkyl esters (eg methyl, ethyl) of the phosphate group.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compound of the invention may be in crystalline form either as the free compound or as a solvate (e.g. hydrate) and it is intended that both forms are within the scope of the present invention. Methods of solvation are generally known within the art.

The oral formulation can be administered to the patient in a therapeutically effective amount. As used herein, a therapeutically effective amount is intended to include at least partially attaining the desired effect, or delaying the onset of, or inhibiting the progression of, or halting or reversing altogether the onset or progression of the proliferative disease or disorder associated with Wnt pathway.

As used herein, the term “effective amount” relates to an amount of compound of Formula (I) which, when administered according to a desired dosing regimen, provides the desired therapeutic activity. Dosing may occur at intervals of minutes, hours, days, weeks, months or years or continuously over any one of these periods. For example, the dosing may occur every other day or once every 3-5 days. Suitable dosages may lie within the range of about 1 μg to about 100 mg per patient in need thereof per dosage, such as in the range of about 1 mg to about 50 mg per patient in need thereof per dosage. In one embodiment, the dosage may be in the range of about 1 mg to about 40 mg, about 1 mg to about 35 mg, about 1 mg to about 30 mg, about 1 mg to about 25 mg or about 1 mg to about 20 mg per patient in need thereof per dosage. In another embodiment, the dosage may be up to about 100 mg, about 80 mg, about 60 mg, about 50 mg, about 40 mg, about 30 mg or about 20 mg per patient in need thereof per dosage.

Suitable dosage amounts and dosing regimens can be determined by the attending physician and may depend on the severity of the condition as well as the general age, health and weight of the patient to be treated.

The oral formulation may be administered in a single dose or a series of doses. The oral formulation may also be administered as a discrete unit or a combination of discrete units.

The formulation may contain other suitable carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The carrier must be pharmaceutically “acceptable” in the sense of being compatible with the other ingredients of the composition and not injurious to the patient. The formulation may conveniently be presented in unit dosage form and may be prepared by any methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then if necessary shaping the product.

While the oral formulation is exemplified as a capsule, other types of oral formulation such as sachets containing a predetermined amount of compound of Formula (I), as a powder or granules, as a solution or a suspension in an aqueous or non-aqueous liquid, or as an oil-in-water liquid emulsion or a water-in-oil liquid emulsion are also within the scope of the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### Examples

Materials: Microcrystalline cellulose (Avicel PH102) was obtained from FMC Biopolymer. Mannitol (Pearlitol 200SD) was obtained from Roquette. Starch (pregelatinised) was obtained from Colorcon. Dicalcium phosphate anhydrous (DCP; Emcompress) was obtained from JRS. Sodium lauryl sulphate (SLS) was obtained from Fisher and magnesium stearate was obtained from Peter Greven. Compound of Formula (I) (API) is used as the active pharmaceutical ingredient and is used in its free base form in all examples.

### Assessment of Compound of Formula (I)

The moisture sorption plot indicates that compound of Formula (I) has about 1% water uptake at above 90% RH.

Dynamic light scattering results shows that the d₁₀, d₅₀, and d₉₀ of compound of Formula (I) is about 2-5 μm, 10-30 μm and 35-55 μm respectively.

In water, compound of Formula (I) has a solubility of about 6 μg/mL. At pH 6, the solubility is about 5 μg/mL. At pH 4, the solubility is about 6.4 μg/mL. At pH 2, the solubility is about 8.4 μg/mL.

The solubility of compound of Formula (I) in various excipients are given in the table below:

| | | | | pH after |
|---|---|---|---|---|
| | Solubility (μg/ml) | | | equilibration, |
| | | | Average | Average |
| Solution/media | Sample-1 | Sample-2 | (n = 2) | (n = 2) |
| Water | 6.00 | 5.97 | 5.99 | 7.95 |
| 2% w/v SLS is water | 457.73 | 429.48 | $43.61 | 8.82 |
| 2% w/v Tween 80 in water | 22.45 | 23.16 | 22.81 | 7.72 |
| 5% w/v PVP K 30 in water | 18.09 | 13.52 | 15.80 | 3.96 |
| 50% w/v Poloxamer 188 in water | 10.86 | 8.39 | 9.63 | 7.23 |
| pH 1.2 (Hydrochloric acid buffer) | 20.81 | 20.87 | 20.84 | — |
| 20% w/v SLSixpB 1.2 buffer | 1861.90 | 2170.12 | 2016.01 | 1.33 |
| 29% w/v Tween 80 in pH 1.2 buffer | 37.29 | 37.09 | 37.19 | 1.25 |
| 5% w/v PVP K 30 in pH 1.2 buffer | 34.83 | 35.22 | 35.03 | 1.25 |
| 50% w/v Poloxamer 188 in | 35.64 | 35.40 | 35.52 | 1.25 |
| pH 1.2 buffer | | | | |
| pH 3.0 (Citrate buffer) | 7.76 | 7.78 | 7.77 | — |
| 28% w/v SL.S in pH 3.0 baffer | 393.73 | 414.95 | 404.34 | 2.95 |
| 2% w/v Tween 80 in pH 3.0 buffer | 23.83 | 26.67 | 25.25 | 3.16 |
| 5% w/v PVP K 30 in pH 3.0 buffer | 19.92 | 15.82 | 17.87 | 3.12 |
| 59% w/v Poloxamer 188 in | 18.72 | 17.85 | 18.28 | 3.30 |
| pH 3.0 buffer | | | | |

In water, compound of Formula (I) shows about 40% degradation after 24 h at 25° C. With the addition of 2% w/v SLS, no (or minimal) degradation was observed after 24 h at 25° C.

### Formulations

Four formulations at high and four at low strength were investigated to examine the effect of different excipients on uniformity and filling characteristics for 1 mg and 20 mg formulations. In these formulations, SLS was included in the formulations as the API is poorly wettable. The addition of SLS also reduces the unpredictable nature of a patient's body constitution. In the dissolution bath the surfactant is not needed as there is SLS in the dissolution media. In the body however, although there are surfactants in the intestine this is not the case in the stomach and the surfactant levels are variable between patients and depending on fed/fasted etc. Therefore in order to ensure good wetting and dissolution SLS was included.

A science of scale tool (SoS) was utilised to determine the drug loading and fill weight for size 4 bovine gelatin capsules. The bulk density of the API and the main excipient were taken in to account for the SoS tool and the final formulations were determined. Four formulations were manufactured and tested for each strength. Simple blend containing the API, a surfactant, an excipient and lubricant was produced.

The formulations are shown in Table 1 and 2.

TABLE 1
Formulations with 20 mg of API.

| | Formulation | Formulation | Formulation | Formulation |
|---|---|---|---|---|
| | A20 | B20 | C20 | D20 |
| Material | Percentage Formula (% w/w) | | | |
| API | 20.00 | 23.53 | 16.67 | 25.00 |
| Pregelatinised | 78.00 | — | — | — |
| Starch 1500 | | | | |
| Mannitol | — | 74.47 | — | — |
| (Pearlitol | | | | |
| 200SD) | | | | |
| Dicalcium | — | — | 81.33 | — |
| Phosphate | | | | |
| anhydrous | | | | |
| Microcrystalline | — | — | — | 73.00 |
| cellulose | | | | |
| (Avicel PH102) | | | | |
| SLS | 1.00 | 1.00 | 1.00 | 1.00 |
| Magnesium | 1.00 | 1.00 | 1.00 | 1.00 |
| stearate | | | | |
| Fill weight (mg) | 100.00 | 85.00 | 120.00 | 80.00 |

TABLE 2
Formulations with 1 mg of API.

| | Formulation | Formulation | Formulation | Formulation |
|---|---|---|---|---|
| | A1 | B1 | C1 | D1 |
| Material | Percentage Formula (% w/w) | | | |
| API | 0.74 | 0.91 | 0.71 | 1.18 |
| Pregelatinised | 97.26 | — | — | — |
| Starch 1500 | | | | |
| Mannitol | — | 91.09 | — | — |
| (Pearlitol | | | | |
| 200SD) | | | | |
| Dicalcium | — | — | 97.29 | — |
| Phosphate | | | | |
| anhydrous | | | | |
| Microcrystalline | — | — | — | 96.82 |
| cellulose | | | | |
| (Avicel PH102) | | | | |
| SLS | 1.00 | 1.00 | 1.00 | 1.00 |
| Magnesium | 1.00 | 1.00 | 1.00 | 1.00 |
| stearate | | | | |
| Fill weight (mg) | 135.00 | 110.00 | 140.00* | 85.00 |

*Fill weight adjusted to 155.00 mg in order to obtain a brim fill. Dose claim changed to 1.11 mg.

### Manufacturing Method

API Properties: The API is cohesive and exhibited poor flow properties. The API was screened through 1000 μm screen along with excipients.

### Process for 20 mg of API—Direct Blending

1. Weighed all excipients and active (pre-screened through a 1000 μm sieve, except magnesium stearate) into suitable containers.
2. Placed excipient in to a suitable container and added all of active and SLS. Blended the material in a Turbula blender for 10 minutes at 49 rpm.
3. Sampled blend from 6 locations in the turbula jar for blend uniformity study.
4. Screened Magnesium stearate through the 250 μm sieve and weighed the required amount. Added a small portion of blend to Magnesium stearate. Added to the remaining blend and mixed for 3 min at 49 rpm in a Turbula.

### Process for 1 mg API-Trituration

1. Weighed all excipients and active (pre-screened through a 1000 μm sieve, except magnesium stearate) into suitable containers.
2. In a suitable sized glass mortar and pestle triturated the materials as per the table below. Used a mixing action rather than a grinding action to prevent the compound of Formula (I) (API) from sticking to the mortar and pestle. Used a brush to remove any adhered material between steps.

| | | Quantity | Mixing |
|---|---|---|---|
| Step | Material | (g) | time |
| 2.1 | Filler | 1 | 30 s |
| 2.2 | API | 0.29 | N/A |
| | SLS | 0.26 | N/A |
| | Filler | 1 | 1 min |
| 2.3 | Filler | 2.5 | 1 min |
| 2.4 | Filler | 5 | 1 min |
| 2.5 | Filler | 10 | 2 min |

3. Transferred the triturated material to a suitable container. Rinsed the mortar and pestle with two aliquots of the remaining excipient. Mixed the aliquots in the mortar using the pestle to remove adhered material and transfer to the blending container. Blended the material in a Turbula blender for 10 minutes at 49 rpm.
4. Sampled blend from 6 locations in the turbula jar for blend uniformity study.
5. Screened Magnesium stearate through the 250 μm sieve and weighed the required amount. Added a small portion of blend to Magnesium stearate. Added to the remaining blend and mixed for 3 min at 49 rpm in a Turbula.

### Blend Uniformity

Using a spatula sampled blend from 6 locations in the turbula jar, as depicted in the diagram below. Placed into glass vial and analysed. Ensured vials are kept upright.

| | | |
|---|---|---|
| TOP | A | B |
| MIDDLE | C | D |
| BOTTOM | E | F |

### Encapsulation

For each blend, a minimum of 20 capsules (¼ of the Torpac Profill 100 capacity) were filled in order to confirm the maximum fill weight for size 4 capsules. When required, tamping was utilised to fit the blend into capsules. Additional blend was also added when the capsules are under filled and the fill weight adjusted accordingly. Thereafter 1 full plate (100 capsules) was filled. Observations including the flow of the blend into capsules and fill volume were assessed during encapsulation.

Encapsulation process as follows:
1. Set up the Torpac Profill size 4.
2. Calculate mean capsule shell weight and target fill weight.
3. Encapsulated via Profill with required amount of blend* per plate by weighing blend in a weigh boat from glass jar using a metal spatula and then pouring on to the Profill. Used the spreader to move the powder in to the capsules. When required, used the tamping pin to compress the powder. (*Includes 1% w/w overage to account for losses on plate)
4. De-dusted by transferring the capsules in appropriate portions into a sieve, switched on vacuum and rolled capsules on the sieve whilst vacuuming underneath.
5. Check weighed all capsules.
6. Rejected all capsules outside of limits. Recorded number of accepted and rejected capsules.

### Larger Scale Formulation Screening

The blends were manufactured at 25 g scale. The blend uniformity data is shown in Tables 3 and 4. The encapsulation data are given in Tables 9 and 10.

TABLE 3
Blend uniformity for 20 mg API formulations

| | Starch | Pearlitol | | Avicel |
|---|---|---|---|---|
| Blend Potency | 1500 | 200SD | DCP | PH102 |
| Blend nominal potency % w/w | 20.20 | 23.77 | 16.84 | 25.25 |
| Blend mean potency % w/w | 20.39 | 24.7 | 17.45 | 25.71 |
| Potency RSD (<6%) | 1.4 | 2.7 | 1.3 | 1.4 |
| Blend potency as % nominal | 100.9 | 103.9 | 103.6 | 101.8 |
| (95-105%) | | | | |

TABLE 4
Blend uniformity for 1 mg API formulations

| | Starch | Pearlitol | | Avicel |
|---|---|---|---|---|
| Blend Potency | 1500 | 200SD | DCP | PH102 |
| Blend nominal potency % w/w | 0.748 | 0.918 | 0,722 | 1.188 |
| Blend mean potency % w/w | 0.75 | 0.93 | 0.72 | 1.2 |
| Potency RSD (<6%) | 1.4 | 3.8 | 3.1 | 2.2 |
| Blend potency as % | 100.3 | 101.3 | 99.7 | 101 |
| nominal (95-105%) | | | | |

All of the formulations show good potency with RSDs within the acceptable range.

### Blend Flow

The flow properties are given in Tables 5 and 6. Magnesium stearate was included to prevent sticking of the blend to the encapsulator contact parts (Torpac).

TABLE 5
Flow properties of 20 mg API formulations

| | Bulk | Tapped | |
|---|---|---|---|
| | Density | Density | Carr's |
| Formulation | (g/mL) | (g/mL) | index |
| A20 | 0.43 | 0.72 | 40.28 |
| B20 | 0.34 | 0.63 | 46.03 |
| C20 | 0.56 | 0.88 | 36.36 |
| D20 | 0.28 | 0.52 | 46.15 |

TABLE 6
Flow properties of 1 mg API formulations

| | Bulk | Tapped | |
|---|---|---|---|
| | Density | Density | Carr's |
| Formulation | (g/mL) | (g/mL) | index |
| A1 | 0.61 | 0.90 | 32.22 |
| B1 | 0.52 | 0.61 | 14.75 |
| C1 | 0.70 | 0.87 | 19.54 |
| D1 | 0.34 | 0.50 | 32.00 |

### Larger Scale Formulation Encapsulation

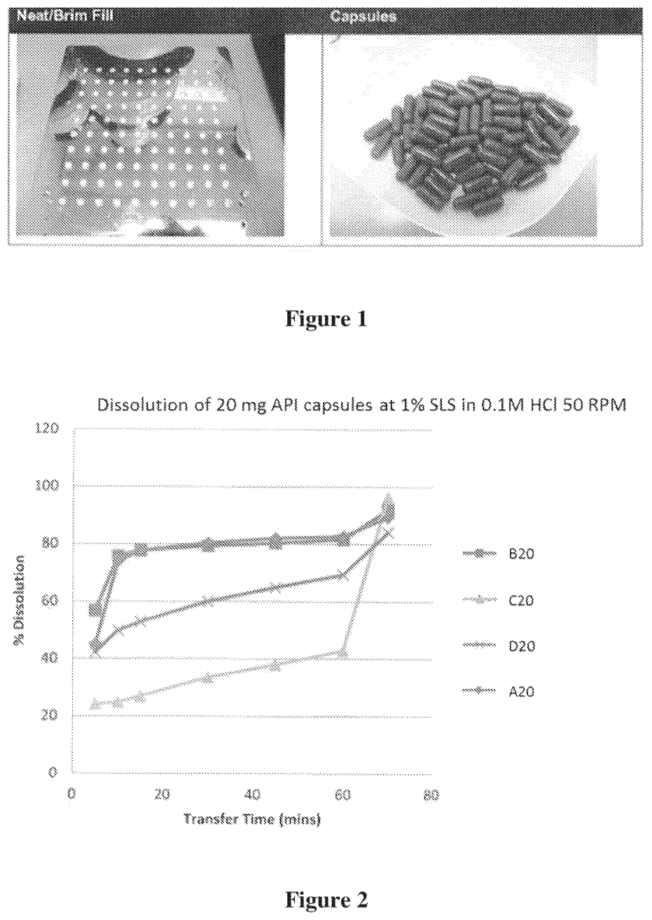

Example of the brim filling technique and the finished capsules are shown in FIG. 1 and the encapsulation data in Table 9 and 10. These data show encapsulation is possible for all formulations. For the 1 mg formulations the yield is good in all cases. For the DCP based 1 mg formulation the capsules were a little underfilled and the formulation can be adjusted accordingly. This was also the case for the 20 mg mannitol formulation.

TABLE 7
Encapsulation data for 20 mg API formulations

| | A20 | B20 | C20 | D20 |
|---|---|---|---|---|
| Encapsulation | Starch 1600 | Mannitol | DCP Anhydrous | Avicel PH102 |
| Filling into capsules | Poor | Poor | Good | Poor |
| Plate fill volume | 1 tamp | 1 tamp, | 0 tamp for | 2 tamps for |
| | for brim | under med | brim fill, | brim fill |
| | fill | by ~24% | 1% overage | |
| | | | for plate | |
| | | | not required | |
| Accepted Capsules | 88 | 82 | 97 | 90 |
| Rejected Capsules | 14 | 18 | 3 | 10 |
| Yield % | 86 | 82 | 97 | 90 |
| Weight RSD % | 3.52 | 5.07 | 2.85 | 5.28 |
| (100 capsules) | 3.52 | 5.07 | | |

TABLE 8
Encapsulation data for 1 mg API formulations

| | A1 | B1 | C1 | D1 |
|---|---|---|---|---|
| Encapsulation | Starch 1500 | Mannitol | DCP Anhydrous | Avicel PH102 |
| Filling into capsules | Acceptable | Good | Good | Good |
| Plate fill volume | 1 light | brim fill | under filled, | 1 tamp for |
| | tamp for | with | more added | brim fill |
| | brim fill | 1 tap | for brim fill | |
| | | | (155 mg/capsules) | |
| Accepted Capsules | 97 | 99 | 98 | 98 |
| Rejected Capsules | 3 | 1 | 2 | 2 |
| Yield % | 97 | 99 | 98 | 98 |
| Weight RSD % | 2.91 | 2.64 | 2.43 | 2.44 |
| (100 capsules) | | | | |

### Assay and Dissolution

Initial dissolution in 0.1 N HCl, 1% SLS at 50 rpm gave the following results for the 20 mg batches: Batches A20, B20 and D20 showed a mean of n=3 greater than 70% after 60 minutes. After increasing the paddles speed to 200 rpm for 10 minutes the % dissolved increased to >90%. For Batch C20 a mean of 52% was achieved after 60 minutes which increased to >90% after increasing the paddle speed (FIG. 2).

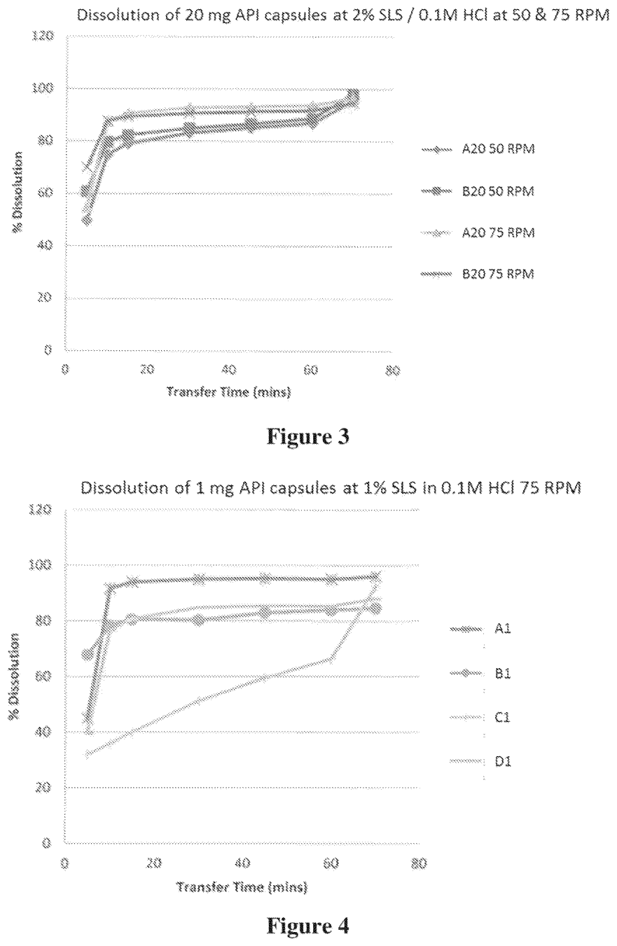

Further development was performed by analysing batches A20 and B20 in 0.1 N HCL 2% SLS at 50 RPM and 75 RPM (FIG. 3). At 50 RPM both batches are greater than 80% after 60 minutes increasing to greater than 90% after 70 minutes. At 75 RPM both batches are >90% after 60 minutes (completed after 30 minutes in some cases) with a slight increase after 70 minutes. Based on these data 2% SLS and 75 rpm was selected as the dissolution media to use in the prototyping stage.

### Assay Data

The assay data for the 1 mg batch is shown in Table 9. All batches are in specification.

TABLE 9
Assay data for 1 mg API formulations

| | Starch | Pearlitol | | Avicel |
|---|---|---|---|---|
| 1 mg | 1500 | 200SD | DCP | PH102 |
| Assay % | 101.1 | 95.5 | 97.8 | 93.0 |
| | (99.6-102.7) | (92.9-98.1) | (96.1-99.5) | (91.0-96.0) |

### Conclusion

During encapsulation, formulation containing DCP Anhydrous flowed better into the capsules in comparison to other three formulations and produced the highest yield of 97%. Flow properties were improved for all 1 mg formulations. All blends were filled successfully into a size 4 capsule with high percentage of yield.

### Formulation Ranking

As the manufacturability and analytical data are variable across the formulations a ranking was developed in order to define the formulations:
Blend uniformity: Must pass
Blend flow: This is relative to the other formulations where 1 is good and 4 poor.
Manufacturability relates to the ease of filling into capsules. This is based on the effort to brim fill (eg tamping) and the yield a scale of 1 (good) to 4 (poor) was used.
Dissolution is the critical quality attribute (CQA). This was rated as 1 (good) to 4 (poor)

Ranking of the formulations (Tables 10 and 11) indicated that both starch and mannitol can be selected as lead formulations. Inclusion of starch 1500 in drug product gave good manufacturability and flow for the 20 mg formulation and chosen as the lead. Mannitol was chosen as the back-up formulation and the absence of SLS will be investigated using this back-up formulation.

TABLE 10
Ranking of 20 mg API formulations

| Ranking of | | | | |
|---|---|---|---|---|
| 20 mg | A20 | B20 | C20 | D20 |
| Formulation | Starch 1500 | Mannitol | DCP Anhydrous | Avicol PH102 |
| Blend Uniformity | Within limits | Within limits | Within limits | Within limits |
| Blend Flow | 2 | 3 | 1 | 3 |
| Manufacturability | 3 | 4 | 1 | 2 |
| Dissolution | 1 | 1 | 4 | 3 |
| Assay | N/A | N/A | N/A | N/A |

TABLE 11
Ranking of 1 mg API formulations

| | A1 | | C1 | D1 |
|---|---|---|---|---|
| Ranking of 1 mg | Starch | B1 | DCP | Avicel |
| Formulation | 1600 | Mannitol | Anhydrous | PH102 |
| Blend Uniformity | Within limits | Within limits | Within limits | Within limits |
| Blond Flow | 3 | 1 | 2 | 3 |
| Manufacturability | 1 | 1 | 1 | 1 |
| Dissolution | 1 | 1 | 4 | 3 |
| Assay | 1 | 2 | 3 | 4 |

### Conclusions on Formulation Selection

Dissolution of the Starch and Mannitol formulations were superior at 1 mg and 20 mg compared to the other formulations. As this is the critical quality attribute, these formulations are chosen for prototyping. Manufacturability issues can be overcome with improved operator technique on larger batches and these formulations are fit for purpose for Phase 1 and the yield is expected to improve.

Based on the final rankings, Starch 1500 was chosen as the lead formulation and Mannitol chosen as back-up formulation.

### Prototype Manufacture

Prototype batches at high and low dose for the lead and back up were produced. The doses were 1 mg and 20 mg in order to span the potential clinical range. The formulations are shown in Table 12-15 and the process in FIGS. 6 and 7. The batches were prepared and analysed.

TABLE 12
Prototype manufacture of 1 mg API formulation containing starch

| Material | Percentage w/w | mg/capsule | g/batch |
|---|---|---|---|
| API | 0.74 | 1.00 | 0.96 |
| Pregelatinised starch | 97.26 | 131.30 | 126.44 |
| (Starch 1500) | | | |
| SLS | 1.00 | 1.35 | 1.30 |
| Magnesium stearate | 1.00 | 1.35 | 1.30 |
| Total | 100.00 | 135.00 | 130.00 |

TABLE 13
Prototype manufacture of 20 mg API
formulation containing starch

| Material | Percentage w/w | mg/capsule | g/batch |
|---|---|---|---|
| API | 20.00 | 20.00 | 26.00 |
| Pregelatinised starch | 78.00 | 78.00 | 101.40 |
| (Starch 1500) | | | |
| SLS | 1.00 | 1.00 | 1.30 |
| Magnesium stearate | 1.00 | 1.00 | 1.30 |
| Total | 100.00 | 100.00 | 130.00 |

TABLE 14
Prototype manufacture of 1 mg API
formulation containing mannitol

| Material | Percentage w/w | mg/capsule | g/batch |
|---|---|---|---|
| API | 0.91 | 1.00 | 1.18 |
| Mannitol | 98.09 | 107.90 | 127.52 |
| (Pearlitol 200SD) | | | |
| Magnesium stearate | 1.00 | 1.10 | 1.30 |
| Total | 100.00 | 110.00 | 130.00 |

TABLE 15
Prototype manufacture of 20 mg API
formulation containing mannitol

| Material | Percentage w/w | mg/capsule | g/batch |
|---|---|---|---|
| API | 23.53 | 20.00 | 30.59 |
| Mannitol | 75.47 | 64.15 | 98.11 |
| (Pearlitol 200SD) | | | |
| Magnesium stearate | 1.00 | 0.85 | 1.30 |
| Total | 100.00 | 85.00 | 130.00 |

### Prototype Results

The t=0 results for the prototype batches are shown in Table 16 to 33. All batches pass from an in-process and final testing point of view. The lead formulation had a yield of 92% for the low dose and 88% for the high dose. The backup had a yield of 99% and 93% respectively. This reflects the flow properties of the two formulations. The blend uniformity (BU) for the lead formulations were slightly tighter than the backup (% RSD) and this is reflected in the content uniformity. All batches pass the prototype test. This may be related to the particle size of the API and the excipients.

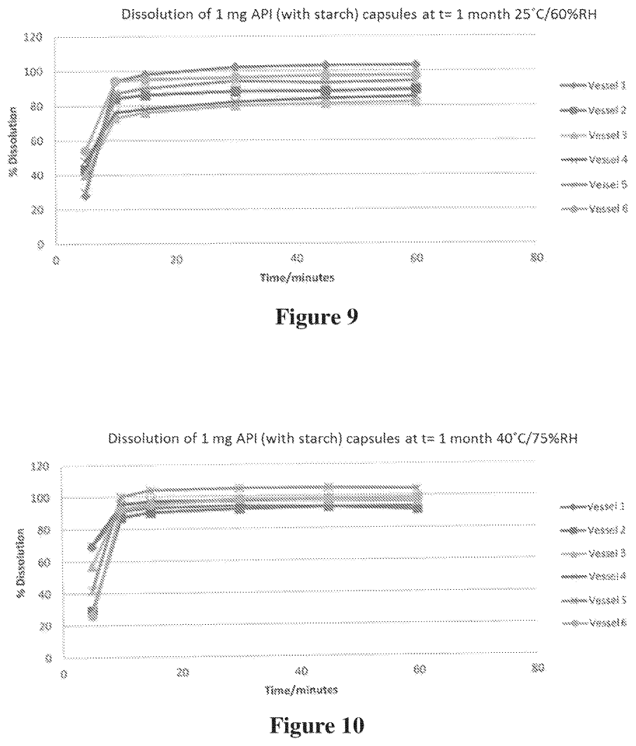

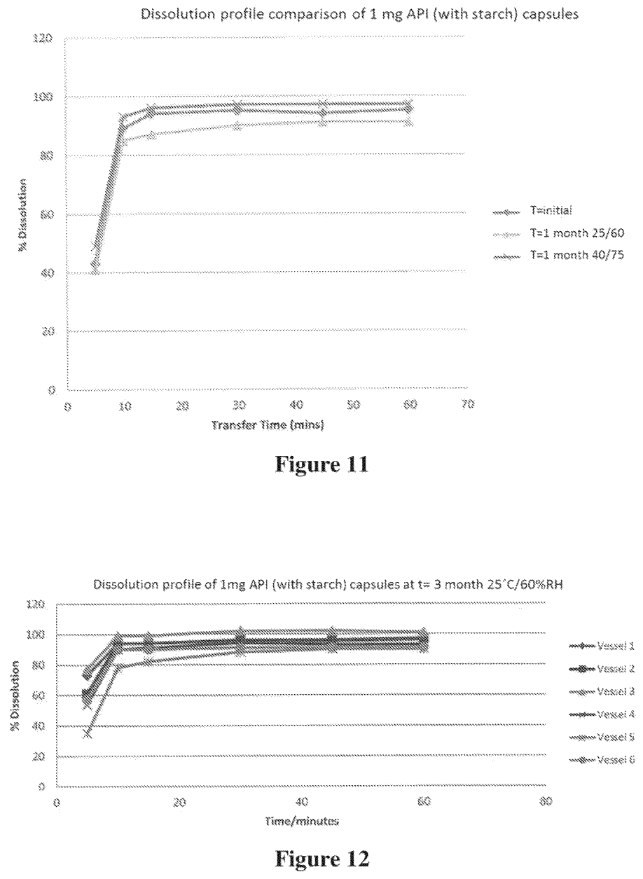

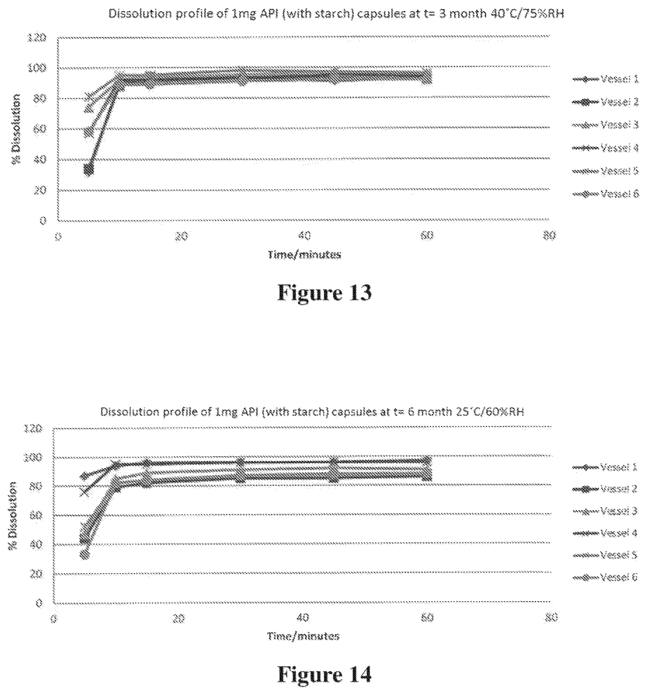

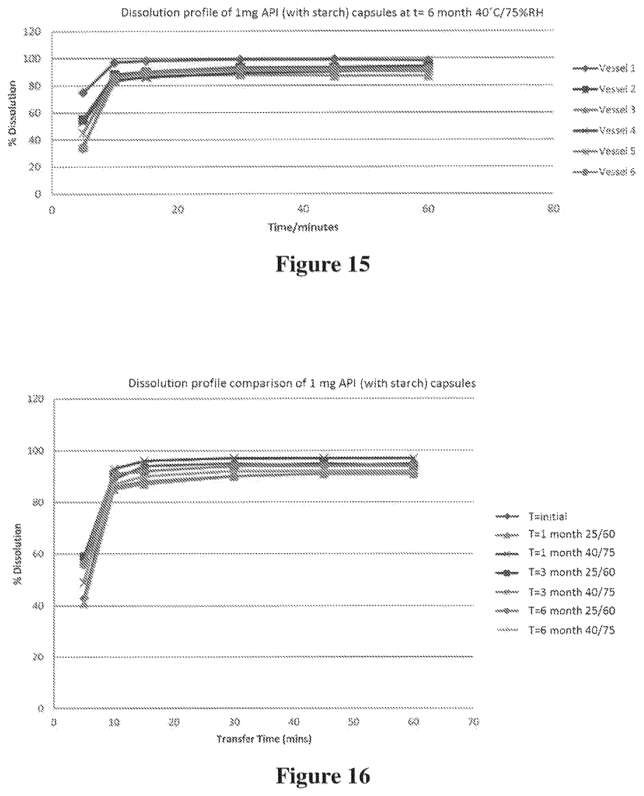

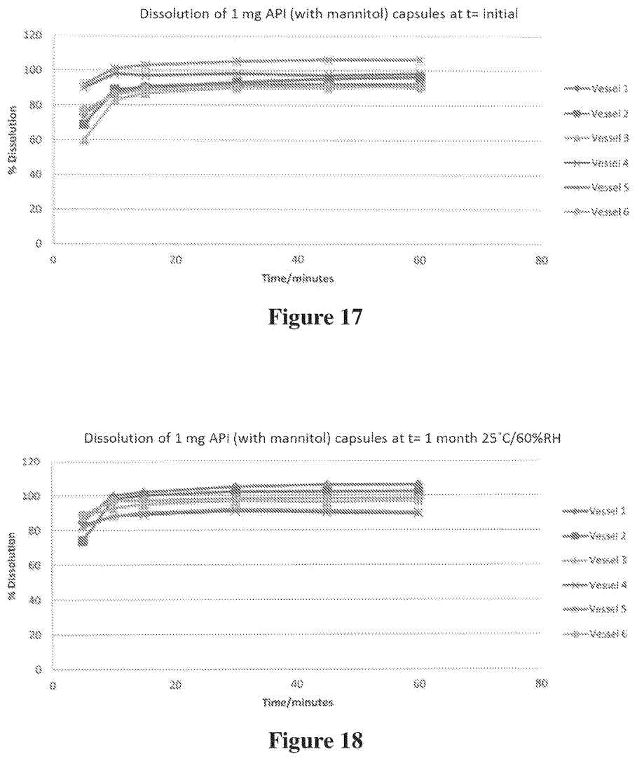

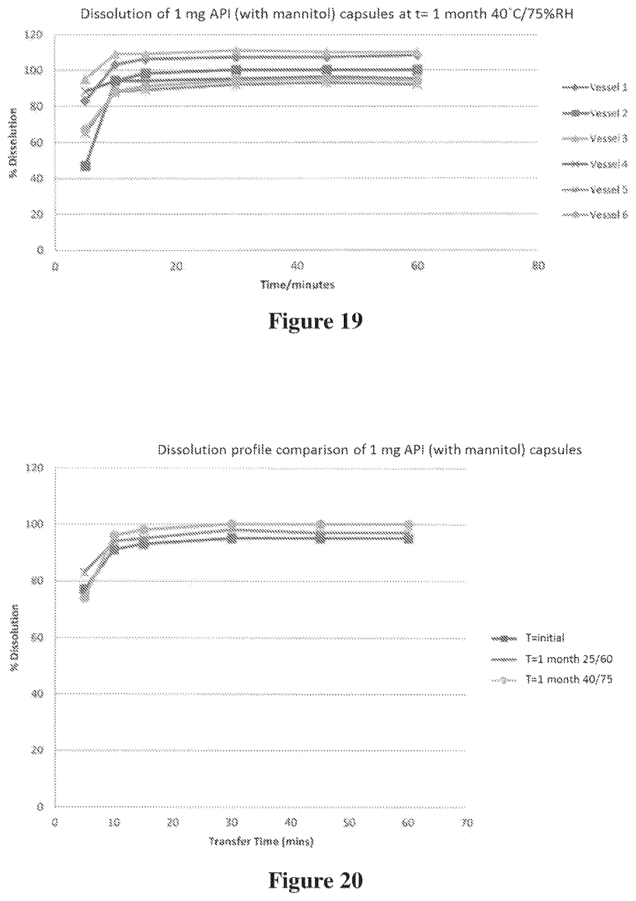

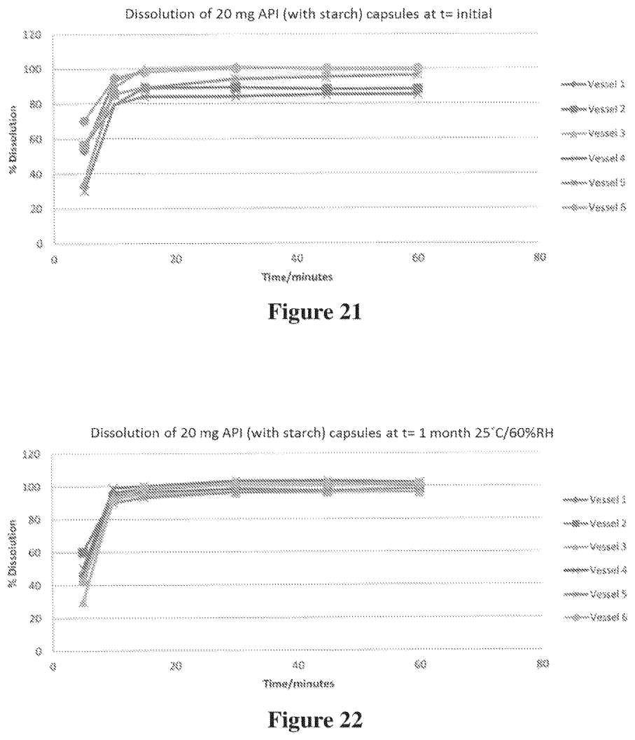

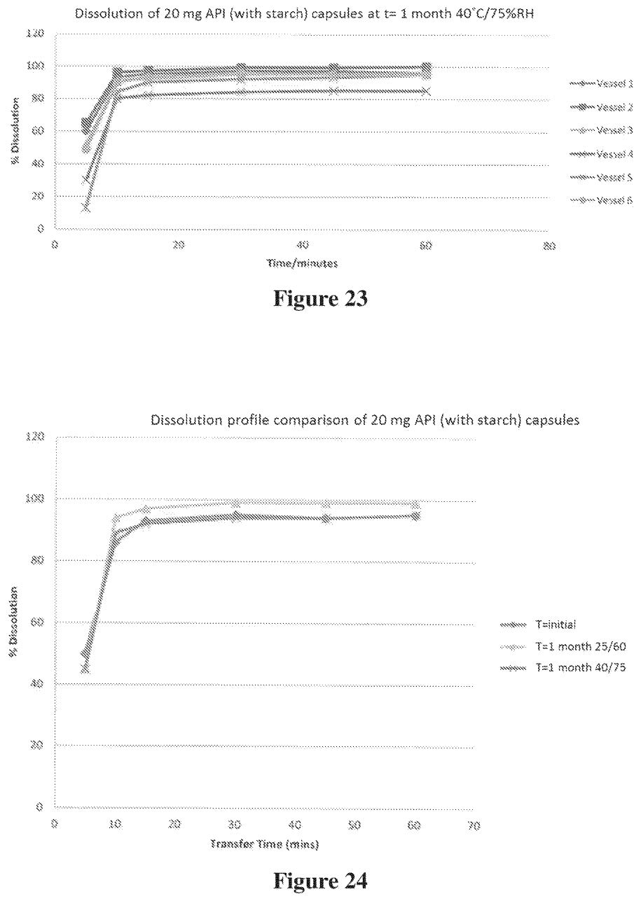

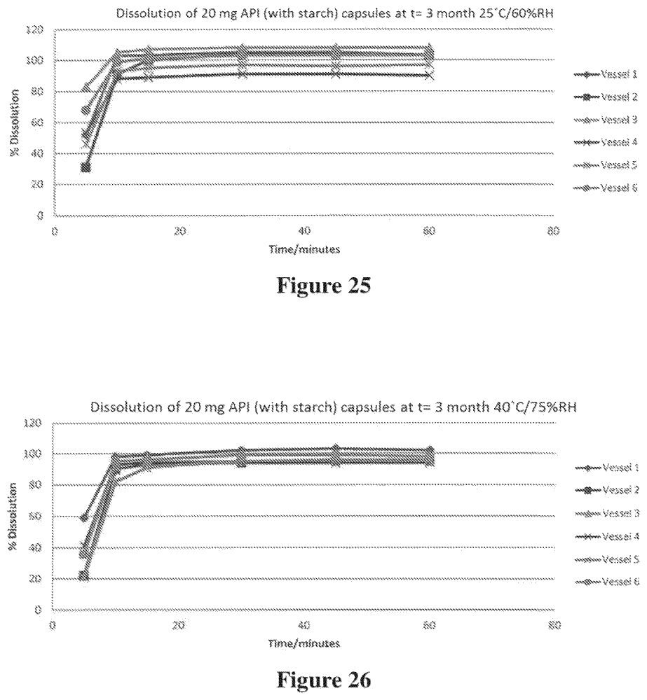

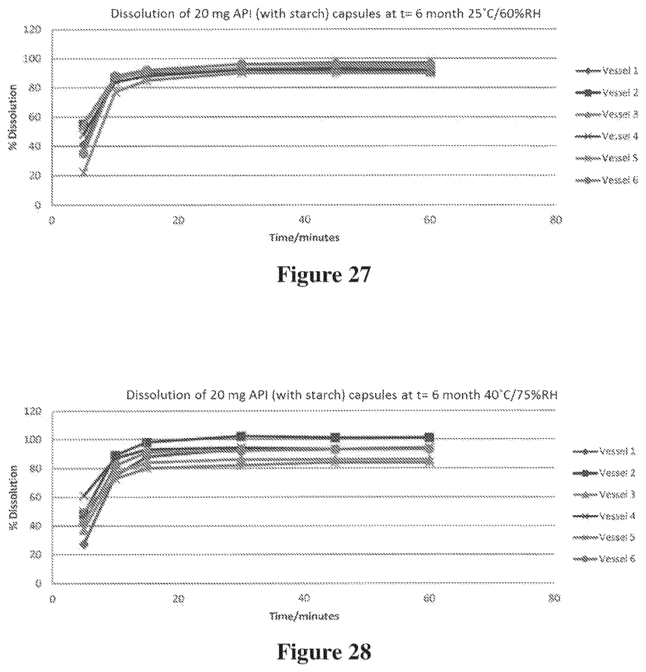

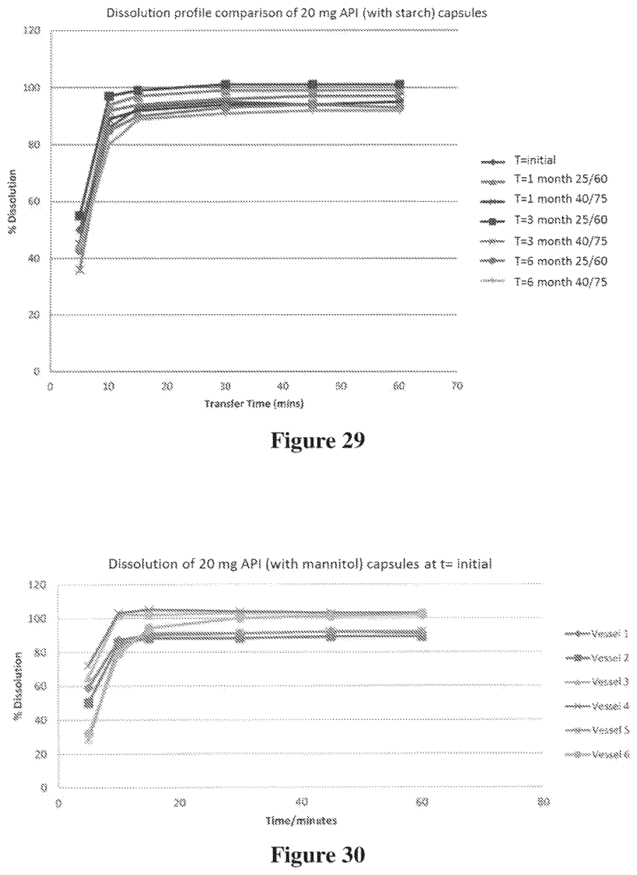

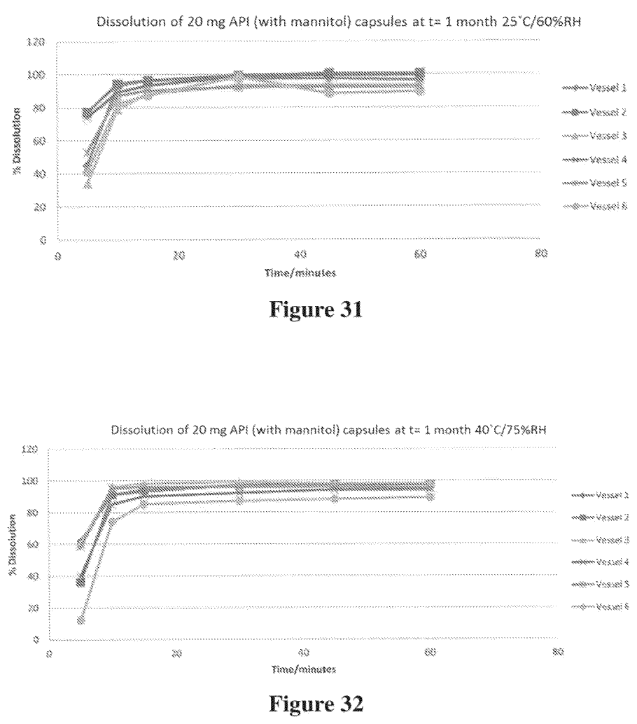

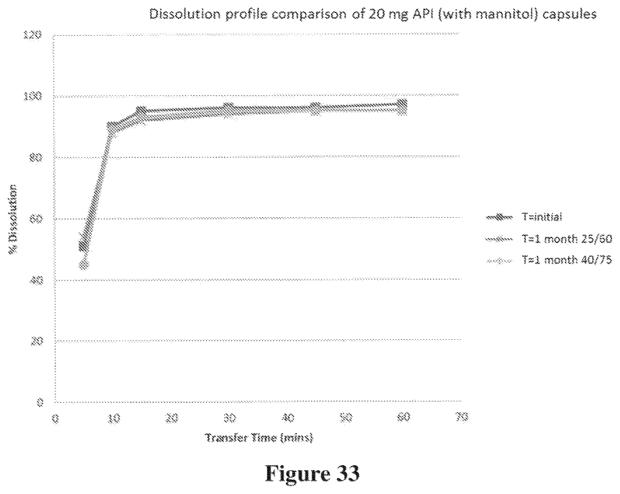

Dissolution is immediate in all cases with dissolution almost complete after 10 minutes. The dissolution is shown in FIG. 8 to 33.

TABLE 16
Data for 1 mg API formulations

| | Starch 1500 | Pearlitol 200SD |
|---|---|---|
| Fill Weight (mg) | 135 | 110 |
| Drug Loading (% w/w ) | 0.74 | 0.91 |
| Blend bulk density g/ml | 0.648 | 0.539 |
| Tapped density g/ml | 0.907 | 0.589 |
| Blend Flow Carr's index % | 28.56 (Poor) | 8.49 (Excellent) |
| Blend nominal potency | 0.748 | 0.918 |
| % w/w | | |
| Blend mean potency % w/w | 0.77 | 0.95 |
| Potency RSD (<6%) | 2.3 | 5.1 |
| Blend potency as % nominal | 102.94 | 103.49 |
| (95-105%) | | |
| Filling into capsules | Poor | Good |
| Plate fill volume | Neat fill | Neat fill |
| | after 2 tamps | |
| Accepted Capsules | 854 | 1088 |
| Rejected Capsules | 76 | 14 |
| Accepted capsules yield % | 92 | 99 |
| Weight RSD % (10 capsules | 2.05-3.96 | 1.28-2.85 |
| per plate checked) | | |
| Content Uniformity | Mean 100.1% | Mean 98.9% |
| | RSD 4.0% | RSD 4.1% |
| | AV 9.6 | AV 9.8 |
| Dissolution after infinity | 95% | 95% |
| run % | | |

TABLE 17
Physical and Chemical testing results of 1 mg API with starch

| | | Moisture |
|---|---|---|
| | Appearance | (% w/w) |
| Specification | Report | Report |
| Initial | Size 4, Swedish orange capsule | 7.8 |
| | containing white powder | |

25° C./60% RH

| | | |
|---|---|---|
| 1 Month | Size 4, Swedish orange capsule | 7.6 |
| | containing white powder | |
| 3 Months | Size 4, Swedish orange capsule | 7.9 |
| | containing white powder | |
| 6 Months | Size 4, Swedish orange capsule | 7.5 |
| | containing white powder | |

40° C./75% RH

| | | |
|---|---|---|
| 1 Month | Size 4, Swedish orange capsule | 8.1 |
| | containing white powder | |
| 3 Months | Size 4, Swedish orange capsule | 8.6 |
| | containing white powder | |
| 6 Months | Size 4, Swedish orange capsule | 8.8 |
| | containing white powder | |

TABLE 18
Assay and related substances results of 1 mg API with starch

| | | Unspecified Related | | | Total |
|---|---|---|---|---|---|
| | | Substances (% Area) | | | Related |
| | Åssay | RRT | RRT | RRT | Substances |
| | (% claim) | 1.02 | 1.36 | 1.43 | (% Area) |
| Specification | Report | Report | | | Report |
| Initial | 99.4 | <0.10 | <0.10 | <0.10 | <0.10 |

25° C./60% RH

| | | | | | |
|---|---|---|---|---|---|
| 1 Month | 98.2 | <0.10 | <0.10 | <0.10 | <0.10 |
| 3 Months | 99.1 | <0.10 | <0.10 | <0.10 | <0.10 |
| 6 Months | 99.6 | <0.10 | <0.10 | <0.10 | <0.10 |

40° C./75% RH

| | | | | | |
|---|---|---|---|---|---|
| 1 Month | 100.0 | <0.10 | <0.10 | <0.10 | <0.10 |
| 3 Months | 96.6 | <0.10 | <0.10 | <0.10 | <0.10 |
| 6 Months | 99.4 | <0.10 | <0.10 | <0.10 | <0.10 |

TABLE 19
Content uniformity results of 1 mg API with starch

| | | | | | Uniformity of Dosage | |
|---|---|---|---|---|---|---|
| | Content Range | | | | Unit (Content Uniformity) | |
| | Min | Max | Mean | RSD | Acceptance | Conforms To |
| | (%) | (%) | (%) | (%) | Value ≤ 15.0 | USP <905> |
| Initial | 93.3 | 105.6 | 100.1 | 4.0 | 9.6 | Complies |

TABLE 20
Dissolution profile of 1 mg API with starch

| | Dissolution profile (% dissolved) | | | | | | | | | | | |
|---|---|---|---|---|---|---|---|---|---|---|---|---|
| | 5 minutes | | 10 minutes | | 15 minutes | | 30 minutes | | 45 minutes | | 60 minutes | |
| Time | Average | Range | Average | Bange | Average | Range | Average | Range | Average | Range | Average | Range |
| Specification | To be Reported | | | | | | | | | | | |
| Initial | 43 | 34-58 | [symbol] | 82-98 | 94 | 88-101 | 95 | 89-100 | [symbol] 4 | 90-99 | 95 | 90-9 [symbol] |

25° C./60% RH

| | | | | | | | | | | | | |
|---|---|---|---|---|---|---|---|---|---|---|---|---|
| 1 Month | 41 | 28- [symbol] 4 | [symbol] | 73-94 | 87 | 76- [symbol] | 90 | 80-102 | [symbol] 1 | 81-103 | [symbol] 1 | 82-103 |
| 3 Months | 59 | 3 [symbol] -77 | [symbol] 1 | 78-99 | 92 | 82-99 | 94 | 88-102 | [symbol] | 90-102 | [symbol] 4 | 90-101 |
| 6 Months | 56 | 33-87 | [symbol] | 79-9 [symbol] | 98 | 82-96 | 90 | 8 [symbol] -96 | [symbol] 1 | 85-96 | [symbol] 1 | 86-97 |

40° C./75% RH

| | | | | | | | | | | | | |
|---|---|---|---|---|---|---|---|---|---|---|---|---|
| 1 Month | 48 | 26-70 | [symbol] | 87-100 | [symbol] 6 | 90-104 | [symbol] 7 | 92-105 | [symbol] 7 | 93-105 | 97 | 91-104 |
| 3 Months | 56 | 32-81 | [symbol] | 88-9 [symbol] | [symbol] 2 | 89-95 | [symbol] 4 | 91-98 | [symbol] 4 | 91-97 | 94 | 92- [symbol] |
| 6 Months | 50 | 34-75 | 87 | 83-97 | [symbol] 0 | 86-98 | [symbol] 2 | 88-99 | [symbol] 2 | 87-99 | [symbol] 2 | 87- [symbol] |

[symbol]

indicates data missing or illegible when filed

TABLE 21
Physical and Chemical testing results of 1 mg API with mannitol

| | | Moisture |
|---|---|---|
| | Appearance | (% ww) |
| Specification | Report | Report |
| Initial | Size 4, Swedish orange capsule | 0.2 |
| | containing white powder | |

25° C./60% RH

| | | |
|---|---|---|
| 1 Month | Size 4, Swedish orange capsule | 0.2 |
| | containing white powder | |

40° C./75% RH

| | | |
|---|---|---|
| 1 Month | Size 4, Swedish orange capsule | 0.2 |
| | containing white powder | |

TABLE 22
Assay and related substances results of 1 mg API with mannitol

| | | Unspecified | |
|---|---|---|---|
| | | Related | |
| | | Substances | Total Related |
| | Assay | (% Area) | Substances |
| | (% claim) | RRT 1.02 | (% Area) |
| Specification | Report | Report | Report |
| Initial | 96.9 | <0.10 | <0.10 |

25° C./60% RH

| | | | |
|---|---|---|---|
| 1 Month | 98.8 | <0.10 | <0.10 |

40° C./75% RH

| | | | |
|---|---|---|---|
| 1 Month | 101.8 | <0.10 | <0.10 |

TABLE 23
Content uniformity results of 1 mg API with mannitol

| | | | | | Uniformity of Dosage | |
|---|---|---|---|---|---|---|
| | Content Range | | | | Unit (Content Uniformity) | |
| | Min | Max | Mean | RSD | Acceptance | Conforms To |
| | (%) | (%) | (%) | (%) | Value ≤ 15.0 | USP <905> |
| Initial | 90.7 | 104. [symbol] | 98.9 | 4.1 | 9.8 | Complies |

[symbol]

indicates data missing or illegible when filed

TABLE 24
Dissolution profile of 1 mg API with mannitol

| | Dissolution profile (% dissolved) | | | | | | | | | | | |
|---|---|---|---|---|---|---|---|---|---|---|---|---|
| | 5 minutes | | 10 minutes | | 15 minutes | | 30 minutes | | 45 minutes | | 60 minutes | |
| Time | Average | Range | Average | Bange | Average | Range | Average | Range | Average | Range | Average | Range |
| Specifi- | | | | | | | | | | | | |
| cation | To be Reported | | | | | | | | | | | |
| Initial | 77 | [symbol] - [symbol] | [symbol] 1 | [symbol] -101 | [symbol] 3 | [symbol] -1 [symbol] | [symbol] | [symbol] -1 [symbol] | [symbol] | [symbol] -1 [symbol] | [symbol] | [symbol] 0-1 [symbol] |

25° C./60% RH

| | | | | | | | | | | | | |
|---|---|---|---|---|---|---|---|---|---|---|---|---|
| 1 Month | [symbol] 3 | [symbol] - [symbol] | [symbol] 4 | [symbol] -100 | [symbol] | [symbol] -102 | [symbol] | [symbol] 1-1 [symbol] | [symbol] | [symbol] -1 [symbol] | [symbol] 7 | [symbol] -1 [symbol] |

40° C./75% RH

| | | | | | | | | | | | | |
|---|---|---|---|---|---|---|---|---|---|---|---|---|
| 1 Month | 74 | [symbol] - [symbol] | [symbol] | [symbol] -1 [symbol] | [symbol] | [symbol] -1 [symbol] | 100 | [symbol] 2-111 | 1 [symbol] | [symbol] -110 | 100 | [symbol] 2-110 |

[symbol]

indicates data missing or illegible when filed

TABLE 25
Data for 20 mg API formulations

| | Starch 1500 | Pearlitol 200SD |
|---|---|---|
| Fill Weight (mg) | 100 | 85 |
| Drug Loading (% w/w ) | 20 | 23.53 |
| Blend bulk density g/ml | 0.452 | 0.425 |
| Tapped density g/ml | 0.705 | 0.64 |
| Blend Flow Carr's index % | 35.89 (Very Poor) | 33.28 (Very Poor) |
| Blend nominal potency | 20.20 | 23.55 |
| % w/w | | |
| Blend mean potency % w/w | 20.54 | 23.93 |
| Potency RSD (<6%) | 1.00 | 2.30 |
| Blend potency as % nominal | 101.68 | 101.61 |
| (95-105%) | | |
| Filling into capsules | Poor | Poor |
| Plate fill volume | Neat Fill after | Neat fill after |
| | 2 tamps | 1 tamp |
| Accepted Capsules | 1053 | 1205 |
| Rejected Capsules | 147 | 95 |
| Accepted capsules yield % | 88 | 93 |
| Weight RSD % (10 capsules | 1.60-3.96 | 0.97-3.17 |
| per plate checked) | | |
| Content Uniformity | Mean 100.2% | Mean 98.7% |
| | RSD 4.2% | RSD 5.2% |
| | AV 10.1 | AV 12.5 |
| Dissolution after infinity | 95% | 95% |
| run % | | |

TABLE 26
Physical and Chemical testing results of 20 mg API with starch

| | | Moisture |
|---|---|---|
| | Appearance | (% w/w) |
| Specification | Report | Report |
| Initial | Size 4, Swedish orange capsule | 6.7 |
| | containing white powder | |

25° C./60% RH

| | | |
|---|---|---|
| 1 Month | Size 4, Swedish orange capsule | 6.4 |
| | containing white powder | |
| 3 Months | Size 4, Swedish orange capsule | 7.1 |
| | containing white powder | |
| 6 Months | Size 4, Swedish orange capsule | 6.1 |
| | containing white powder | |

40° C./75% RH

| | | |
|---|---|---|
| 1 Month | Size 4, Swedish orange capsule | 7.2 |
| | containing white powder | |
| 3 Months | Size 4, Swedish orange capsule | 7.7 |
| | containing white powder | |
| 6 Months | Size 4, Swedish orange capsule | 8.6 |
| | containing white powder | |

TABLE 27
Assay and related substances results of 20 mg API with starch

| | | Unspecified | |
|---|---|---|---|
| | | Related | |
| | | Substances | Related |
| | Assay | (% Area) | Substances |
| | (% claim) | RRT 1.02 | (% Area) |
| Specification | Report | Report | Report |
| Initial | 103.5 | <0.10 | <0.10 |

25° C./60% RH

| | | | |
|---|---|---|---|
| 1 Month | 101.5 | <0.10 | <0.10 |
| 3 Months | 96.7 | <0.10 | <0.10 |
| 6 Months | 98.1 | <0.10 | <0.10 |

40° C./75% RH

| | | | |
|---|---|---|---|
| 1 Month | 100.4 | <0.10 | <0.10 |
| 3 Months | 98.3 | <0.10 | <0.10 |
| 6 Months | 99.8 | <0.10 | <0.10 |

TABLE 28
Content uniformity results of 20 mg API with starch

| | | | | | Uniformity of Dosage | |
|---|---|---|---|---|---|---|
| | Content Range | | | | Unit (Content Uniformity) | |
| | Min | Max | Mean | RSD | Acceptance | Conforms To |
| | (%) | (%) | (%) | (%) | Value ≤ 15.0 | USP <905> |
| Initial | 93.6 | 105.7 | 100.2 | 4.2 | 10.1 | Complies |

TABLE 29
Dissolution profile of 20 mg API with starch

| | Dissolution profile (% dissolved) | | | | | | | | | | | |
|---|---|---|---|---|---|---|---|---|---|---|---|---|
| | 5 minutes | | 10 minutes | | 15 minutes | | 30 minutes | | 45 minutes | | 60 minutes | |
| Time | Average | Range | Average | Range | Average | Range | Average | Bange | Average | Range | Average | Range |
| Specification | To be Reported | | | | | | | | | | | |
| | 50 | 30-70 | [symbol] | 79-94 | [symbol] | 84-100 | 9 [symbol] | 84-104 | [symbol] 4 | 85-100 | 95 | 85-100 |

25° C./60% RH

| | | | | | | | | | | | | |
|---|---|---|---|---|---|---|---|---|---|---|---|---|
| 1 Month | 45 | 30-60 | [symbol] 4 | 90- [symbol] | 97 | [symbol] 8-100 | [symbol] | 96-10 [symbol] | [symbol] | [symbol] -10 [symbol] | [symbol] | [symbol] -102 |
| 3 Months | 55 | 31-83 | 97 | 88-106 | 99 | [symbol] -107 | 101 | 91-1 [symbol] | 101 | [symbol] 1-1 [symbol] | 101 | [symbol] -10 [symbol] |
| 6 Months | 4 [symbol] | 22- [symbol] | [symbol] | 77- [symbol] | 90 | 85- [symbol] 2 | [symbol] | [symbol] 0- [symbol] | [symbol] | 90-97 | [symbol] | [symbol] 0- [symbol] 7 |

40° C./75% RH

| | | | | | | | | | | | | |
|---|---|---|---|---|---|---|---|---|---|---|---|---|
| 1 Month | 45 | 13- [symbol] | 89 | [symbol] 0-96 | 82 | 82-97 | 94 | 84-99 | [symbol] 4 | 85-99 | 95 | 85-100 |
| 3 Months | 36 | 21-59 | [symbol] 2 | 82-98 | [symbol] 4 | 91-99 | [symbol] | 94-102 | [symbol] 7 | 94-103 | 97 | 94-102 |
| 6 Months | 44 | 27-61 | 80 | 73-89 | 89 | 80-98 | [symbol] 1 | 82-102 | [symbol] 2 | 84-101 | [symbol] 2 | 84-101 |

[symbol]

indicates data missing or illegible when filed

TABLE 30
Physical and Chemical testing results of 20 mg API with mannitol

| | | Moisture |
|---|---|---|
| | Appearance | (% ww) |
| Specification | Report | Report |
| Initial | Size 4, Swedish orange capsule | 0.2 |
| | containing white powder | |

25° C./60% RH

| | | |
|---|---|---|
| 1 Month | Size 4, Swedish orange capsule | 0.2 |
| | containing white powder | |

40° C./75% RH

| | | |
|---|---|---|
| 1 Month | Size 4, Swedish orange capsule | 0.2 |
| | containing white powder | |

TABLE 31
Assay and related substances results of 20 mg API with mannitol

| | | Unspecified | |
|---|---|---|---|
| | | Related | |
| | | Substances | Total Related |
| | Assay | (% Area) | Substances |
| | (% claim) | RRT 1.02 | (% Area) |
| Specification | Report | Report | Report |
| Initial | 99.3 | <0.10 | <0.10 |

25° C./60% RH

| | | | |
|---|---|---|---|
| 1 Month | 97.7 | <0.10 | <0.10 |

40° C./75% RH

| | | | |
|---|---|---|---|
| 1 Month | 99.5 | <0.10 | <0.10 |

TABLE 32
Content uniformity results of 20 mg API with mannitol

| | | | | | Uniformity of Dosage | |
|---|---|---|---|---|---|---|
| | Content Range | | | | Unit (Content Uniformity) | |
| | Min | Max | Mean | RSD | Acceptance | Conforms To |
| | (%) | (%) | (%) | (%) | Value ≤ 15.0 | USP <905> |
| Initial | 89.6 | 106.8 | [symbol] 8.7 | 6.2 | 12. [symbol] | Complies |

[symbol]

indicates data missing or illegible when filed

TABLE 33
Dissolution profile of 20 mg API with mannitol

| | Dissolution profile (% dissolved) | | | | | | | | | | | |
|---|---|---|---|---|---|---|---|---|---|---|---|---|
| | 5 minutes | | 10 minutes | | 15 minutes | | 30 minutes | | 45 minutes | | 60 minutes | |
| Time | Average | Range | Average | Bange | Average | Range | Average | Range | Average | Range | Average | Range |
| Specification | To be Reported | | | | | | | | | | | |
| Initial | [symbol] 1 | 2 [symbol] -72 | [symbol] | 7 [symbol] -10 [symbol] | [symbol] | [symbol] -10 [symbol] | [symbol] | [symbol] -104 | [symbol] | [symbol] -10 [symbol] | [symbol] 7 | [symbol] -10 [symbol] |

25° C./60% RH

| | | | | | | | | | | | | |
|---|---|---|---|---|---|---|---|---|---|---|---|---|
| 1 Month | [symbol] 4 | [symbol] 4-77 | [symbol] | 7 [symbol] - [symbol] 4 | [symbol] 2 | [symbol] 7- [symbol] | [symbol] 4 | [symbol] - [symbol] | [symbol] | [symbol] -100 | [symbol] | [symbol] -100 |

40° C./75% RH

| | | | | | | | | | | | | |
|---|---|---|---|---|---|---|---|---|---|---|---|---|
| 1 Month | [symbol] | 12- [symbol] 2 | [symbol] | 74- [symbol] | [symbol] | [symbol] - [symbol] | [symbol] | [symbol] 7- [symbol] | [symbol] | [symbol] - [symbol] | [symbol] | [symbol] - [symbol] |

[symbol]

indicates data missing or illegible when filed

### Analytical Release Data for the Prototype Batches

### 1 mg Lead

Mean assay with 95.0 to 105. % and no related substances obtained >0.1%
Content uniformity data passes the USP acceptance criteria of <15.0. The mean is within 1% of the assay data.
Dissolution profile is good and complete after 30 minutes and all six capsules >85% after 30 minutes. The range of the six capsules is comparable to the range obtained with the CU.
Water content is 7.8% and has been established using the volumetric technique. Although high the amount is comparable to the amount of water present in the main excipient starch.

### 1 mg Back-Up

Mean assay with 95.0 to 105.% and no related substances obtained >0.1%.
Content uniformity data passes the USP acceptance criteria of <15.0. The mean is within 2% of the assay data.
Dissolution profile is good and complete after 30 minutes and all six capsules >85% after 30 minutes. The range of the six capsules is comparable to the range obtained with the CU.
Water content is 0.2% and has been established using the Coulometric technique. Although low the amount is comparable to the amount of water present in the main excipients Mannitol.

### 20 mg Lead

Mean assay with 95.0 to 105. % and no related substances obtained >0.1%.
Content uniformity data passes the USP acceptance criteria of <15.0. The mean is within 3% of the assay data.
Dissolution profile is good and complete after 45 minutes and all six capsules >85% after 45 minutes. The range of the six capsules is more variable than other batches when comparing the range obtained with the CU. For the one capsule obtained at 85% the capsule weight for this was also the lowest capsule weight and if we adjust for the capsule weight the value would be in the region of 90%.
Water content is 6.7% and has been established using the volumetric technique. This is lower than the 1 mg batch would be expected as there is an increase in the amount of API and therefore a decrease in the amount of starch.

### 20 mg Back-Up

Mean assay with 95.0 to 105. % and no related substances obtained >0.1%
Content uniformity data passes the USP acceptance criteria of <15.0. The mean is within 1% of the assay data.
Dissolution profile is good and complete after 45 minutes and all six capsules >85% after 45 minutes. The range of the six capsules is comparable to the range obtained with the CU.
Water content is 0.2% and has been established using the Coulometric technique and is comparable to the 1 mg back up batch.

### CONCLUSION

Two prototype formulations, at 1 mg and 20 mg, of API as well as a manufacturing process suitable for Clinical Trials Manufacture have been developed. The in process data and release data show the formulations meet specification and are suitable for Phase 1. Based on the 1 month stability data, the presence of SLS can mitigate risk in dealing with the poor solubility of the API. Further, based on the results of B1 in FIGS. 3 and 4, while the SLS was added to the dissolution medium, the overall results show that the presence of SLS helps in the dissolution profile of the API.

Two strengths (1 mg and 10 mg) containing starch have further been tested and the formulations are shown in Tables 34 and 35.

TABLE 34
1 mg API formulation

| Material | Percentage w/w | mg/capsule |
|---|---|---|
| API | 0.74 | 1.00 |
| Pregelatinised starch | 97.26 | 131.30 |
| (Starch 1500) | | |
| SLS | 1.00 | 1.35 |
| Magnesium stearate | 1.00 | 1.35 |
| Total | 100.00 | 135.00 |

TABLE 35
10 mg API formulation

| Material | Percentage w/w | mg/capsule |
|---|---|---|
| API | 8.33 | 10.00 |
| Pregelatinised starch | 89.67 | 107.60 |
| (Starch 1500) | | |
| SLS | 1.00 | 1.20 |
| Magnesium stearate | 1.00 | 1.20 |
| Total | 100.00 | 120.00 |

1: An oral formulation, comprising:
a) a compound of Formula (I) having the following structure: 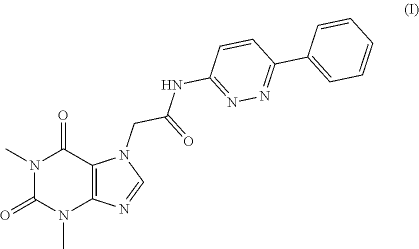 [SMILES: Cn1c(=O)c2c(ncn2CC(=O)Nc2ccc(-c3ccccc3)nn2)n(C)c1=O]
or a pharmaceutically acceptable salt or solvate thereof; and
b) at least one excipient selected from the group consisting of starch, mannitol, dicalcium phosphate and microcrystalline cellulose; and
c) sodium lauryl sulphate;
wherein the oral formulation is provided as a capsule.

2: The oral formulation according to claim 1, wherein the at least one excipient is from about 70% w/w to about 99% w/w.

3: The oral formulation according to claim 1, wherein the at least one excipient is mannitol or starch.

4: The oral formulation according to claim 1, wherein the compound of Formula (I) is from about 0.5% w/w to about 27% w/w.

5: The oral formulation according to claim 1, wherein sodium lauryl sulphate is from about 0.5% w/w to about 3% w/w.

6: The oral formulation according to claim 1, further comprising magnesium stearate.

7: The oral formulation according to claim 6, wherein the magnesium stearate is from about 0.5% w/w to about 2% w/w.

8: The oral formulation according to claim 1, comprising:
a) a compound of Formula (I) having the structure: 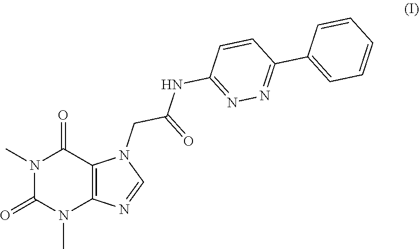 [SMILES: Cn1c(=O)c2c(ncn2CC(=O)Nc2ccc(-c3ccccc3)nn2)n(C)c1=O]
or a pharmaceutically acceptable salt or solvate thereof from about 0.5% w/w to about 27% w/w;
b) mannitol from about 69% w/w to about 98.5% w/w;
c) sodium lauryl sulphate from about 0.5% w/w to about 2% w/w; and
d) magnesium stearate from about 0.5% w/w to about 2% w/w;
wherein the oral formulation is provided as a capsule.

9: The oral formulation according to claim 1, comprising:
a) a compound of Formula (I) having the structure: 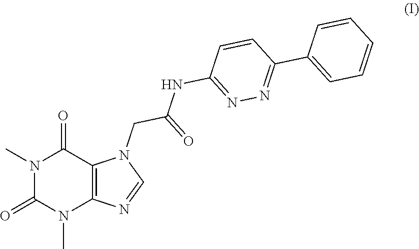 [SMILES: Cn1c(=O)c2c(ncn2CC(=O)Nc2ccc(-c3ccccc3)nn2)n(C)c1=O]
or a pharmaceutically acceptable salt or solvate thereof from about 0.5% w/w to about 27% w/w;
b) starch from about 69% w/w to about 98.5% w/w;
c) sodium lauryl sulphate from about 0.5% w/w to about 2% w/w; and
d) magnesium stearate from about 0.5% w/w to about 2% w/w;
wherein the oral formulation is provided as a capsule.

10: A process for manufacturing an oral formulation, the oral formulation comprising:
a) a compound of Formula (I) having the following structure: 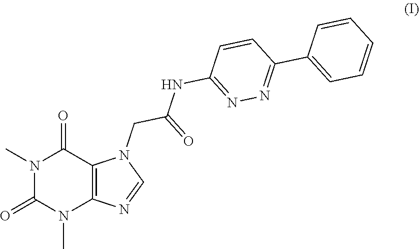 [SMILES: Cn1c(=O)c2c(ncn2CC(=O)Nc2ccc(-c3ccccc3)nn2)n(C)c1=O]
or a pharmaceutically acceptable salt or solvate thereof;
b) at least one excipient selected from the group consisting of starch, mannitol, dicalcium phosphate and microcrystalline cellulose; and
c) sodium lauryl sulphate;
wherein the oral formulation is provided as a capsule;
the process including the steps of:
i) mixing compound of Formula (I), the at least one excipient and sodium lauryl sulphate to form a mixture; and
ii) encapsulating the mixture in a capsule to form the oral formulation.

11: The process according to claim 10, wherein the mixing step is selected from direct blending or trituration.

12: The process according to claim 10, wherein the encapsulating step includes encapsulating the mixture into gelatin capsules.

13: A method of treating a proliferative disease or disorder associated with Wnt pathway in a subject in need thereof, comprising administering an oral formulation to the subject, the oral formulation, comprising:
a) a compound of Formula (I) having the following structure: 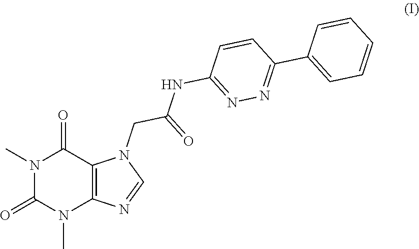 [SMILES: Cn1c(=O)c2c(ncn2CC(=O)Nc2ccc(-c3ccccc3)nn2)n(C)c1=O]
or a pharmaceutically acceptable salt or solvate thereof;
b) at least one excipient selected from the group consisting of starch, mannitol, dicalcium phosphate and microcrystalline cellulose; and
c) sodium lauryl sulphate;
wherein the oral formulation is provided as a capsule.

14: The method according to claim 13, wherein the proliferative disease or condition is selected from the group consisting of cancer, fibrosis, stem cell and diabetic retinopathy, rheumatoid arthritis, psoriasis and myocardial infarction.

15: The method according to claim 13, wherein the proliferative disease or condition is selected from the group consisting of cervical cancer, colon cancer, breast cancer, bladder cancer, head and neck cancer, gastric cancer, lung cancer, ovarian cancer, prostate cancer, thyroid cancer, non-small-cell lung cancer, chronic lymphocytic leukemia, mesothelioma, melanoma, pancreatic adenocarcinoma, basal cell carcinoma, osteosarcoma, hepatocellular carcinoma, Wilm's tumour, medulloblastoma, pulmonary fibrosis, liver fibrosis, skin fibrosis, renal fibrosis, stem cell and diabetic retinopathy, rheumatoid arthritis, psoriasis and myocardial infarction.

16-21. (canceled)
